(19) United States
(12) Patent Application Publication
(10) Pub. No.: US 2025/0177754 A1
(43) Pub. Date: Jun. 5, 2025

(54) MEDICAL DEVICE AND METHOD FOR DELIVERING CARDIAC PACING PULSES

(71) Applicant: Medtronic, Inc., Minneapolis, MN (US)

(72) Inventors: Lonny V. CABELKA, San Clemente, CA (US); Marshall J. RASMUSSEN, Mesa, AZ (US); Jon E. ZIMMER, Shoreview, MN (US)

(21) Appl. No.: 18/842,377

(22) PCT Filed: Feb. 24, 2023
(86) PCT No.: PCT/IB2023/051726

§ 371 Date: Aug. 28, 2024

Related U.S. Application Data
(60) Provisional application No. 63/316,741, filed on Mar. 4, 2022.

(51) Int. Cl. A61N 1/365; A61N 1/05; A61N 1/39

(52) U.S. Cl. CPC A61N 1/365; A61N 1/0563; A61N 1/3912; A61N 1/3918; A61N 1/39622; A61N 1/3981

(57) ABSTRACT

A medical device is configured to deliver a cardiac pacing pulse by enabling a bypass circuit to couple a cardiac pacing voltage source to a cardiac pacing output pathway that excludes a first portion of a high voltage output circuit used to deliver cardioversion/defibrillation shock pulses by the medical device and includes a second portion of the high voltage output circuit used for delivering cardioversion/defibrillation shock pulses.

### TECHNICAL FIELD

The disclosure relates generally to a medical device and method for delivering cardiac pacing pulses.

### BACKGROUND

Medical devices may sense electrophysiological signals from the heart, brain, nerve, muscle or other tissue. Such devices may be implantable, wearable or external devices using implantable and/or surface (skin) electrodes for sensing the electrophysiological signals. In some cases, such devices may be configured to deliver a therapy based on the sensed electrophysiological signals. For example, implantable or external cardiac pacemakers, cardioverter defibrillators, cardiac monitors and the like, sense cardiac electrical signals from a patient's heart. The medical device may sense cardiac electrical signals from the heart and deliver electrical stimulation therapies, such as cardiac pacing pulses and/or cardioversion or defibrillation (CV/DF) shocks, to the heart using electrodes, which may be carried by medical electrical leads extending from the medical device to position electrodes within or near the patient's heart.

A cardiac pacemaker or cardioverter defibrillator may deliver therapeutic electrical stimulation to the heart via electrodes carried by one or more medical electrical leads coupled to the medical device. Cardiac signals sensed from the heart may be analyzed for detecting an abnormal rhythm. Upon detection of an abnormal rhythm, such as bradycardia, tachycardia or fibrillation, an appropriate electrical stimulation pulse or pulses may be delivered to restore or maintain a more normal rhythm of the heart. For example, an implantable cardioverter defibrillator (ICD) may deliver bradycardia pacing pulses to the heart of the patient in the absence of sensed intrinsic myocardial depolarization signals, e.g., R-waves, deliver anti-tachycardia pacing pulses in response to detecting tachycardia, or deliver CV/DF shocks to the heart upon detecting tachycardia or fibrillation.

### SUMMARY

In general, the disclosure is directed to a medical device and techniques for delivering cardiac pacing pulses using high surface area, low impedance electrodes. The medical device may be a pacemaker or ICD configured to deliver cardiac pacing pulses using extra-cardiac electrodes, e.g., electrodes carried by non-transvenous leads or transvenous leads positioned in an extra-cardiac location. A medical device operating according to the techniques disclosed herein may generate cardiac pacing pulses that can be delivered via a high surface area, low impedance electrode vector by delivering a pacing pulse via a high voltage output circuit that includes first switching circuitry used for delivering high voltage CV/DF shocks that is bypassed for delivery of the pacing pulse. The high voltage output circuit includes second switching circuitry providing a return path for either CV/DF shocks or cardiac pacing pulses delivered via the high surface area, low impedance electrodes. The first switching circuitry and the second switching circuitry of the output circuit may be enabled during delivery of high voltage CV/DF shock pulses.

The first switching circuitry can be disabled by a control circuit of the medical device and bypassed during delivery of a cardiac pacing pulse by enabling a bypass circuit that couples a cardiac pacing voltage source to a portion of the high voltage output circuit. In some examples, the medical device may deliver CV/DF shocks using the same high surface area, low impedance electrode vector used for delivering cardiac pacing pulses by charging a high voltage holding capacitor for delivering the CV/DF shock via the high voltage output circuit via a different output pathway, which includes the first switching circuitry, than the cardiac pacing output pathway, which excludes the first switching circuitry.

In one example, the disclosure provides a medical device including a high voltage therapy circuit, a cardiac pacing voltage source and a bypass circuit. The high voltage therapy circuit includes a high voltage capacitor chargeable to a shock voltage amplitude, a high voltage charging circuit configured to charge the high voltage capacitor to the shock voltage amplitude for generating a cardioversion/defibrillation shock pulse; and a high voltage output circuit including a first portion configured to couple the high voltage capacitor to a first electrode terminal and a second portion configured to couple the high voltage capacitor to a second electrode terminal for delivering the cardioversion/defibrillation shock pulse. The cardiac pacing voltage source is configured to generate a cardiac pacing pulse signal having a pacing voltage amplitude that is less than the shock voltage amplitude. The bypass circuit is configured to couple the cardiac pacing voltage source to a cardiac pacing output pathway that excludes the first portion of the high voltage output circuit and includes the second portion of the high voltage output circuit for delivering the cardiac pacing pulse signal via the first electrode terminal and the second electrode terminal.

In another example, the disclosure provides a method that includes generating a cardiac pacing pulse signal by a cardiac pacing voltage source of a therapy delivery circuit of the medical device and enabling a bypass circuit of the medical device to couple the cardiac pacing voltage source to a cardiac pacing output pathway. The cardiac pacing output pathway excludes a first portion of a high voltage output circuit of the therapy delivery circuit configured to couple a high voltage capacitor of the therapy delivery circuit to a first electrode terminal for delivering a cardioversion/defibrillation shock pulse. The cardiac pacing output pathway includes a second portion of the high voltage output circuit configured to couple the high voltage capacitor to a second electrode terminal used for delivering the cardioversion/defibrillation shock pulse. The method further includes delivering the cardiac pacing pulse signal via the first electrode terminal and the second electrode terminal.

In yet another example, the disclosure provides a non-transitory computer readable medium storing a set of instructions that, when executed by a control circuit of a medical device, cause the medical device to generate a cardiac pacing pulse signal by a cardiac pacing voltage source of a therapy delivery circuit of the medical device and enable a bypass circuit of the medical device to couple the cardiac pacing voltage source to a cardiac pacing output pathway. The cardiac pacing output pathway excludes a first portion of a high voltage output circuit of the therapy delivery circuit configured to couple a high voltage capacitor of the therapy delivery circuit to a first electrode terminal for delivering a cardioversion/defibrillation shock pulse. The cardiac pacing output pathway includes a second portion of the high voltage output circuit configured to couple the high voltage capacitor to a second electrode terminal used for delivering the cardioversion/defibrillation shock pulse. The instructions further cause the medical device to deliver the cardiac pacing pulse signal via the first electrode terminal and the second electrode terminal.

Further disclosed herein is the subject matter of the following examples:

Example 1. A medical device comprising a therapy delivery circuit that includes a high voltage therapy circuit including a high voltage capacitor, a high voltage charging circuit and a high voltage output circuit. The high voltage capacitor is chargeable to a shock voltage amplitude. The high voltage charging circuit is configured to charge the high voltage capacitor to the shock voltage amplitude for generating a cardioversion/defibrillation shock pulse. The high voltage output circuit includes a first portion configured to couple the high voltage capacitor to a first electrode terminal and a second portion configured to couple the high voltage capacitor to a second electrode terminal for delivering the cardioversion/defibrillation shock pulse. The therapy delivery circuit further includes a cardiac pacing voltage source configured to generate a cardiac pacing pulse signal having a pacing voltage amplitude that is less than the shock voltage amplitude. The therapy delivery circuit further includes a bypass circuit configured to couple the cardiac pacing voltage source to a cardiac pacing output pathway that excludes the first portion of the high voltage output circuit and includes the second portion of the high voltage output circuit for delivering the cardiac pacing pulse signal via the first electrode terminal and the second electrode terminal.

Example 2. The medical device of example 1 further comprising a sensing circuit configured to sense at least one cardiac signal and a control circuit in communication with the sensing circuit and the therapy delivery circuit, the control circuit configured to determine a need for cardiac pacing based on the at least one cardiac signal and control the therapy delivery circuit to deliver the cardiac pacing pulse signal by enabling the bypass circuit to couple the cardiac pacing voltage source to the cardiac pacing output pathway in response to determining the need for cardiac pacing.

Example 3. The medical device of any of examples 1-2, wherein the first portion of the high voltage output circuit includes a first high operating current switching device between a positive terminal of the high voltage capacitor and the first electrode terminal and a second high operating current switching device between the positive terminal of the high voltage capacitor and the second electrode terminal. The second portion of the high voltage output circuit includes a third switching device between the first electrode terminal and a negative terminal of the high voltage capacitor and a fourth switching device between the second electrode terminal and the negative terminal of the high voltage capacitor. The bypass circuit is configured to couple the cardiac pacing voltage source to the cardiac pacing output pathway that excludes the first portion of the high voltage output circuit comprising the first switching device and the second switching device.

Example 4. The medical device of any of examples 2-3, wherein the bypass circuit includes at least one bypass switching device and the control circuit is further configured to enable the bypass circuit by controlling the at least one bypass switching device to conduct the cardiac pacing pulse signal to the cardiac pacing output pathway.

Example 5. The medical device of any of examples 2-4, wherein the bypass circuit includes a first channel including at least a first switching device and a second channel including at least a second switching device. The control circuit is further configured to selectively enable the first switching device of the first channel or the second switching device of the second channel to conduct the cardiac pacing pulse signal to one of the first electrode terminal or the second electrode terminal, respectively, for bypassing the first portion of the high voltage output circuit.

Example 6. The medical device of any of examples 1-5, wherein the high voltage charging circuit is further configured to generate a rail voltage by charging the high voltage capacitor to a voltage less than the shock voltage amplitude, and the cardiac pacing voltage source further includes a voltage regulator configured to receive the rail voltage and generate the cardiac pacing pulse signal as a voltage regulated output signal. The bypass circuit is further configured to, when enabled, couple the voltage regulator to the cardiac pacing output pathway.

Example 7. The medical device of any of examples 1-5, wherein the cardiac pacing voltage source further comprises at least one charge pump for generating the cardiac pacing pulse signal. The bypass circuit is configured to couple the cardiac pacing voltage source to the cardiac pacing output pathway by coupling the at least one charge pump to the cardiac pacing output pathway.

Example 8. The medical device of any of examples 1-7, wherein the cardiac pacing voltage source further includes a first voltage source configured to generate a first cardiac pacing pulse having up to a first maximum voltage amplitude of a first range of pacing pulse voltage amplitudes and a second voltage source configured to generate a second cardiac pacing pulse signal having up to a second maximum voltage amplitude of a second range of pacing pulse voltage amplitudes, the second maximum voltage amplitude being greater than the first maximum voltage amplitude. The bypass circuit is further configured to couple the cardiac pacing voltage source of the therapy delivery circuit to the cardiac pacing output pathway by selectively coupling one of the first voltage source or the second voltage source to the cardiac pacing output pathway.

Example 9. The medical device of example 8, wherein the first voltage source includes a low voltage capacitor chargeable to the first maximum voltage of the first range of pacing pulse voltage amplitudes and a low voltage charging circuit configured to charge the low voltage capacitor up to the first maximum voltage amplitude of the first range of pacing pulse voltage amplitudes. When the first voltage source is selected for delivering the first cardiac pacing pulse signal, the bypass circuit is further configured to selectively couple the first voltage source of the cardiac pacing voltage source to the cardiac pacing output by coupling the low voltage capacitor to the cardiac pacing output pathway.

Example 10. The medical device of any of examples 8-9, wherein the bypass circuit includes a first channel and a second channel. The first channel can include a first switching device and a second switching device. The second switching may be coupled to the first electrode terminal. The second channel can include a third switching device and a fourth switching device The fourth switching device may be coupled to the second electrode terminal. The medical device may further include a control circuit that is configured to establish a cardiac pacing pulse voltage amplitude, compare the cardiac pacing pulse voltage amplitude to the first range of pacing pulse voltage amplitudes and the second range of pacing pulse voltage amplitudes, and select one of the first voltage source and the second voltage source based on the cardiac pacing pulse voltage amplitude falling into one of the respective first range of pacing pulse voltage amplitudes and the second range of pacing pulse voltage amplitudes. In response to selecting the first voltage source, the control circuit is configured to enable one of the second switching device of the first channel or the fourth switching device of the second channel to conduct the first cardiac pacing voltage signal to the respective one of the first terminal or the second terminal. In response to selecting the second voltage source, the control circuit is configured to enable one of: (a) the first switching device and the second switching device of the first channel, or (b) the third switching device and the fourth switching device of the second channel to conduct the second cardiac pacing voltage signal to the respective one of the first electrode terminal or the second electrode terminal.

Example 11. The medical device of any of examples 8-10, wherein the second voltage source includes one of a voltage regulator or a series of at least two charge pumps.

Example 12. The medical device of any of examples 2-11, wherein the control circuit is further configured to detect a tachyarrhythmia based on the at least one sensed cardiac signal. Responsive to the control circuit detecting the tachyarrhythmia, the high voltage therapy circuit is further configured to charge the high voltage charging circuit to the shock voltage amplitude for generating the cardioversion/defibrillation shock pulse and enable the first portion and the second portion of the high voltage output circuit to deliver the cardioversion/defibrillation shock pulse.

Example 13. The medical device of any of examples 1-12, wherein the first electrode terminal is couplable to a first high surface area electrode and the second terminal is couplable to a second high surface area electrode. The first and second high surface area electrodes may be carried by an extra-cardiac lead.

Example 14. A method that can be performed by a medical device includes generating a cardiac pacing pulse signal by a cardiac pacing voltage source of a therapy delivery circuit of the medical device, enabling a bypass circuit of the medical device to couple the cardiac pacing voltage source to a cardiac pacing output pathway. The cardiac pacing output pathway excludes a first portion of a high voltage output circuit of the therapy delivery circuit configured to couple a high voltage capacitor of the therapy delivery circuit to a first electrode terminal for delivering a cardioversion/defibrillation shock pulse. The cardiac pacing output pathway includes a second portion of the high voltage output circuit configured to couple the high voltage capacitor to a second electrode terminal used for delivering the cardioversion/defibrillation shock pulse. The method further includes delivering the cardiac pacing pulse signal via the first electrode terminal and the second electrode terminal.

Example 15. The method of example 14, further including sensing by a sensing circuit at least one cardiac signal and determining by a control circuit of the medical device a need for cardiac pacing based on the at least one sensed cardiac signal. The method further includes, in response to determining the need for cardiac pacing, enabling the bypass circuit by the control circuit to couple the cardiac pacing voltage source of the therapy delivery circuit to the cardiac pacing output pathway.

Example 16. The method of any of examples 14-15 further including enabling the bypass circuit to couple the cardiac pacing voltage source to the cardiac pacing output pathway by excluding the first portion by excluding, from the cardiac pacing output pathway, a first high operating current switching device between a positive terminal of a high voltage capacitor of the therapy delivery circuit and a first electrode terminal and a second high operating current switching device between the positive terminal of the high voltage capacitor and a second electrode terminal. The high voltage capacitor is chargeable to a shock voltage amplitude. The method further includes including the second portion by including, in the cardiac pacing output pathway, at least one of a third switching device between the first electrode terminal and a negative terminal of the high voltage capacitor and a fourth switching device between the second electrode terminal and the negative terminal of the high voltage capacitor.

Example 17. The method of any of examples 14-16, wherein enabling the bypass circuit includes controlling at least one switching device of the bypass circuit to conduct the cardiac pacing pulse signal to the cardiac pacing output pathway.

Example 18. The method of any of examples 14-17, wherein enabling the bypass circuit further includes selectively enabling by a control circuit of the medical device at least one switching device of one of a first channel of the bypass circuit or a second channel of the bypass circuit to conduct the cardiac pacing pulse signal via the at least one switching device to one of the first electrode terminal or the second electrode terminal, respectively, for bypassing the first portion of the high voltage output circuit.

Example 19. The method of any of examples 14-18 further including generating the cardiac pacing pulse signal by generating a rail voltage by charging the high voltage capacitor to a voltage less than the shock voltage amplitude, receiving the rail voltage by a voltage regulator, and generating the cardiac pacing pulse signal as a voltage regulated output signal of the voltage regulator. The method may further include enabling the bypass circuit of the medical device to couple the cardiac pacing voltage source to the cardiac pacing output pathway by enabling the bypass circuit to couple the voltage regulator to the cardiac pacing output pathway.

Example 20. The method of any of examples 14-18 further including generating the cardiac pacing pulse signal by at least one charge pump of the therapy delivery circuit and enabling the bypass circuit to couple the cardiac pacing voltage source to the cardiac pacing output pathway by coupling the at least one charge pump to the cardiac pacing output pathway.

Example 21. The method of any of examples 14-20, wherein generating the cardiac pacing pulse signal includes one of: generating, by a first voltage source of the cardiac pacing voltage source, the cardiac pacing pulse signal as a first cardiac pacing pulse signal having up to a first maximum voltage amplitude of a first range of pacing pulse voltage amplitudes or generating, by a second voltage source of the cardiac pacing voltage source, the cardiac pacing pulse signal as a second cardiac pacing pulse signal having up to a second maximum voltage amplitude of a second range of pacing pulse voltage amplitudes. The second maximum voltage amplitude being greater than the first maximum voltage amplitude. The method further including enabling the bypass circuit to couple the cardiac pacing voltage source to the cardiac pacing output pathway by selectively coupling one of the first voltage source or the second voltage source to the cardiac pacing output pathway.

Example 22. The method of example 21, further including selecting one of the first voltage source or the second voltage source for generating the cardiac pacing pulse signal. When the first voltage source is selected, the method includes generating the first cardiac pacing pulse signal by the first voltage source by charging a low voltage capacitor of the therapy delivery circuit up to the first maximum voltage amplitude of the first range of pacing pulse voltage amplitudes and selectively coupling, by the bypass circuit, the first voltage source of the cardiac pacing voltage source to the cardiac pacing output by coupling the low voltage capacitor to the cardiac pacing output pathway.

Example 23. The method of any of examples 21-22, wherein the bypass circuit includes a first channel including a first switching device and a second switching device. The second switching device being coupled to a first electrode terminal. The bypass circuit further includes a second channel having a third switching device and a fourth switching device. The fourth switching device being coupled to a second electrode terminal. The method further includes establishing a cardiac pacing pulse voltage amplitude, comparing the cardiac pacing pulse voltage amplitude to the first range of pacing pulse voltage amplitudes and the second range of pacing pulse voltage amplitudes, and selecting one of the first voltage source and the second voltage source based on the cardiac pacing pulse voltage amplitude falling into one of the respective first range of pacing pulse voltage amplitudes or the second range of pacing pulse voltage amplitudes. When the first voltage source is selected, enabling one of the second switching device of the first channel or the fourth switching device of the second channel to conduct the first cardiac pacing voltage signal to the respective one of the first electrode terminal or the second electrode terminal. When the second voltage source is selected, enabling one of: (a) the first switching device and the second switching device of the first channel, or (b) the third switching device and the fourth switching device of the second channel to conduct the second cardiac pacing voltage signal to the respective one of the first electrode terminal or the second electrode terminal.

Example 24. The method of any of examples 21-23 wherein the second voltage source includes one of a voltage regulator or a series of at least two charge pumps.

Example 25. The method of any of examples 15-24 further including detecting a tachyarrhythmia based on the at least one sensed cardiac signal, charging the high voltage capacitor of the therapy delivery circuit to a shock voltage amplitude for generating a cardioversion/defibrillation shock pulse, and enabling the first portion and the second portion of the high voltage output circuit to deliver the cardioversion/defibrillation shock pulse.

Example 26. The method of any of examples 14-25 wherein delivering the cardiac pacing pulse signal further includes delivering the cardiac pacing pulse via the first electrode terminal coupled to a first high surface area electrode and the second terminal coupled to a second high surface area electrode. The first and second high surface area electrodes can be carried by an extra-cardiac lead.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIGS. 1A and 1B are conceptual diagrams of one example of an ICD system that may be configured to sense cardiac event signals, detect arrhythmia and deliver electrical stimulation therapy according to the techniques disclosed herein.

FIGS. 2A-2C are conceptual diagrams of a patient implanted with an ICD system in a different implant configuration than the arrangement shown in FIGS. 1A-1B.

FIG. 3 is a conceptual diagram of an ICD according to one example.

FIG. 4 is a conceptual diagram of circuitry that can be included in therapy delivery circuit of FIG. 3 according to some examples.

FIG. 5 is a diagram of a bypass circuit that may be included in the therapy delivery circuit of FIG. 4 according to some examples.

FIG. 6 is a conceptual diagram of a therapy delivery circuit according to another example.

FIG. 7 is a conceptual diagram of a therapy delivery circuit according to yet another example.

FIG. 8 is a flow chart of a method for delivering cardiac pacing pulses by an ICD according to some examples.

### DETAILED DESCRIPTION

In general, this disclosure describes medical devices and techniques for delivering cardiac pacing pulses using relatively high surface area, low impedance electrodes that may be implanted in an extra-cardiac or extra-cardiovascular location. The high surface area electrodes may be used for delivering CV/DF shocks by the medical device by enabling a high voltage output circuit pathway for discharging a high voltage capacitor. A portion of the high voltage output circuit can be used for delivering the cardiac pacing pulses via the high surface area electrodes by using a bypass circuit that excludes a first portion of the high voltage output circuit and enables cardiac pacing pulses to be delivered via the high surface area electrode terminals and a second portion of the high voltage output circuit.

As used herein, the term “extra-cardiac” refers to a position outside the heart and may refer to a position outside of the pericardium surrounding the heart of a patient. Extra-cardiac electrodes may be carried by a non-transvenous lead or a transvenous lead. A transvenous extra-cardiac lead may carry implantable electrodes that can be positioned intravenously but outside the heart in an extra-cardiac location, e.g., within the internal thoracic vein, jugular vein, or another vein. As used herein, the term “extra-cardiovascular”refers to a position outside the blood vessels and heart, which may also be outside the pericardium surrounding the heart of a patient. Implantable electrodes carried by non-transvenous, extra-cardiovascular leads may be positioned extra-thoracically (outside the ribcage and sternum) or intra-thoracically (beneath the ribcage or sternum) but may not be in intimate contact with myocardial tissue.

As disclosed herein, a medical device includes a therapy delivery circuit including operative circuitry configured to deliver high voltage CV/DF shock pulses using high surface area, low impedance electrodes. The medical device is further configured to generate relatively lower voltage cardiac pacing pulses that are delivered, using a portion of the high voltage therapy delivery circuitry, to a high surface area, low impedance electrode that may also be used for delivering CV/DF shock pulses. As described below, the therapy delivery circuitry of an ICD may include a bypass circuit for bypassing a portion of a high voltage output circuit that requires high operating current for controlling delivery of high voltage CV/DF shocks.

When cardiac pacing pulse delivery is needed, a portion of the high voltage output circuit that requires high operating current is disabled and bypassed to enable the therapy delivery circuit to deliver relatively lower voltage cardiac pacing pulses via the high surface area electrodes. When a CV/DF shock is needed, the bypass circuit is disabled and the high voltage output circuit is enabled such that the high voltage CV/DF shocks can be delivered via a conducting state of components of the high voltage output circuit that may require a high operating current. By bypassing one or more high operating current components of the high voltage output circuit during cardiac pacing pulse delivery, cardiac pacing pulses having relatively lower voltage amplitude than CV/DF shocks can be delivered efficiently via a high surface area, low impedance electrode in a manner that uses less current from the ICD power source and effectively captures the heart.

The techniques disclosed herein may be implemented in any implantable or external or wearable pacemaker or ICD and particularly in a pacemaker or ICD having extra-cardiac electrodes. The electrodes may be carried by an implantable medical electrical lead extending from the pacemaker or ICD and/or carried by the housing of the pacemaker or ICD. The techniques disclosed herein are not necessarily limited to implantable systems, however, and may be implemented in an external pacemaker or ICD using cutaneous surface electrodes or transcutaneous electrodes.

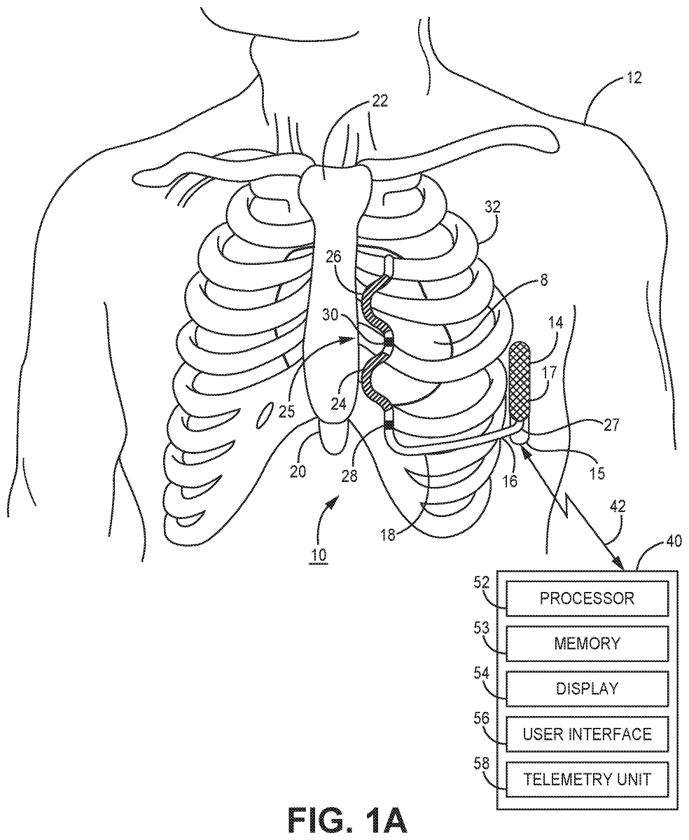

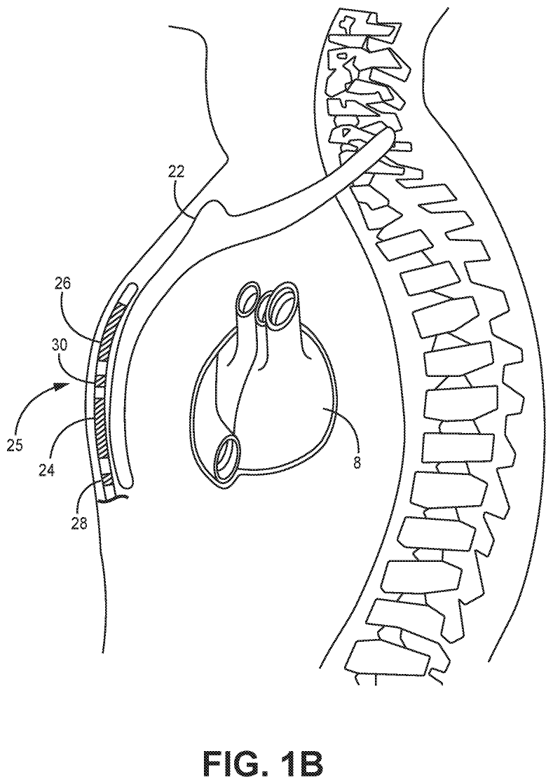

FIGS. 1A and 1B are conceptual diagrams of one example of an ICD system 10 that may be configured to sense cardiac electrical signals, detect arrhythmia and deliver electrical stimulation therapy according to the techniques disclosed herein. FIG. 1A is a front view of ICD system 10 implanted within patient 12. FIG. 1B is a side view of ICD system 10 implanted within patient 12. ICD system 10 includes an ICD 14 connected to an electrical stimulation and sensing lead 16, positioned in an extra-cardiovascular location in this example. FIGS. 1A and 1B are described in the context of an ICD system 10 capable of providing high voltage CV/DF shocks and/or cardiac pacing pulses in response to detecting a cardiac arrhythmia based on processing of sensed cardiac electrical signals. The techniques disclosed herein for delivering cardiac electrical stimulation therapies may be implemented in a variety of medical devices including external, transcutaneous, or implantable cardiac pacemakers and ICDs.

ICD 14 includes a housing 15 that forms a hermetic seal that protects internal components of ICD 14. The housing 15 of ICD 14 may be formed of a conductive material, such as titanium or titanium alloy. The housing 15 may function as an electrode (sometimes referred to as a “can” electrode). Housing 15 may be used as an active can electrode for use in delivering CV/DF shocks or other high voltage pulses delivered using a high voltage therapy circuit. In other examples, housing 15 may be available for use in delivering unipolar, relatively lower voltage cardiac pacing pulses and/or for sensing cardiac electrical signals in combination with electrodes carried by lead 16. In other instances, the housing 15 of ICD 14 may include a plurality of electrodes on an outer portion of the housing. The outer portion(s) of the housing 15 functioning as an electrode(s) may be coated with a material, such as titanium nitride, e.g., for reducing post-stimulation polarization artifact.

ICD 14 includes a connector assembly 17 (also referred to as a connector block or header) that includes electrical feedthroughs crossing housing 15 to provide electrical connections between conductors extending within the lead body 18 of lead 16 and electronic components included within the housing 15 of ICD 14. As will be described in further detail herein, housing 15 may house one or more processing circuits, memories, transceivers, cardiac electrical signal sensing circuitry, therapy delivery circuitry, power sources and other components for sensing cardiac electrical signals, detecting a heart rhythm, and controlling and delivering electrical stimulation pulses to treat an abnormal heart rhythm.

Elongated lead body 18 has a proximal end 27 that includes a lead connector (not shown) configured to be connected to ICD connector assembly 17 and a distal portion 25 that includes one or more electrodes. In the example illustrated in FIGS. 1A and 1B, the distal portion 25 of lead body 18 includes high surface area, low impedance electrodes 24 and 26 and relatively low surface area, higher impedance electrodes 28 and 30. Electrodes 24 and 26 are elongated electrodes that may extend along a portion of the length of lead body 18 to form a relatively high surface area, low impedance electrode that can be used for delivering high voltage CV/DF pulses. A CV shock pulse may be synchronized to an intrinsic R-wave sensed by ICD 14 for terminating non-sinus, tachycardia. A DF shock pulse may be delivered without synchronization to a sensed R-wave for terminating fibrillation. In either case, the high voltage, high energy CV/DF shock pulse is delivered to the heart using high surface area electrodes, e.g., elongated coil electrodes, to cause depolarization of a large mass of the myocardial tissue simultaneously. The simultaneous depolarization of the large mass of myocardial tissue is followed by repolarization and an associated state of physiological refractoriness of the large mass, which disrupts the conduction of aberrant depolarizations through the heart that are causing the tachyarrhythmia. In this way, the tachyarrhythmia may be successfully terminated because the heart's normal, intrinsic electrical conduction system (or a cardiac pacing pulse) may initiate the next heartbeat to restore a more normal, organized propagation and conduction of the myocardial depolarizations through the heart.

High surface area electrodes, such as electrodes 24 and 26 and/or housing 24, are used to deliver CV/DF shocks in order to encompass a large mass of the heart within the electrical field between the electrodes selected in the CV/DF electrode vector and to avoid tissue injury at the electrode sites that could occur when delivering high voltage shocks via a lower electrode surface area, resulting in a high current density at a more localized tissue site. Electrodes 24 and 26 may be configured to be activated concurrently to form one, large surface area, low impedance anode or cathode. Alternatively, electrodes 24 and 26 may form separate high surface area, low impedance electrodes in which case each of the electrodes 24 and 26 may be activated independently, e.g., as an anode or cathode, for delivering CV/DF shock pulses.

As disclosed herein, electrodes 24 and 26 may be selected for delivering cardiac pacing pulses, having a much lower voltage amplitude than a CV/DF shock but may be a higher voltage than the voltage amplitude required of cardiac pacing pulses delivered using endocardial or epicardial pacing electrodes. One electrode 24 or 26 may serve as a pacing cathode with the other electrode 26 or 24 serving as the return anode. In other examples, one electrode 24 or 26, or concurrently selected electrodes 24 and 26, may serve as the pacing cathode with the housing 15 or another available electrode serving as the return anode electrode.

For the sake of convenience, electrodes 24 and 26 are referred to herein as “coil electrodes” because they may take the form of a coiled electrode (which may include a single wire or filar or multiple wires or filars, e.g., a braided multi-filar wire, a stranded multi-filar wire, etc.) winding around a longitudinal portion of lead body 18 to provide a relatively high surface area for delivering high voltage CV/DF shocks. However, it is to be understood that electrodes 24 and 26 may be configured as other types of high surface area electrodes that can be used for delivering CV/DF shocks, which may include ribbon electrodes, plate electrodes, serpentine electrodes, zig-zagging electrodes, or other types of physical electrode configurations that provide a relatively large surface area and low impedance and do not necessarily include a coiled wire.

Coil electrodes 24 and 26 (and in some examples housing 15) are sometimes referred to as “defibrillation electrodes” or “CV/DF electrodes” because they are utilized, individually or collectively, for delivering high voltage CV/DF shocks. However, as disclosed herein coil electrodes 24 and 26 (and in some examples housing 15) may be utilized in a cardiac pacing electrode vector to provide cardiac pacing pulse delivery. Furthermore, in some examples, coil electrodes 24 and 26 may be utilized in a sensing electrode vector for providing sensing functionality in addition to being utilized for delivering high voltage CV/DF shocks and/or cardiac pacing pulses. In this sense, the use of the term “defibrillation electrode” or “CV/DF electrode” herein should not be considered as limiting the electrodes 24 and 26 for use in only high voltage CV/DF shock therapy applications. For example, either of coil electrodes 24 and 26 may be used as a sensing electrode in a sensing electrode vector for sensing cardiac electrical signals and determining a need for an electrical stimulation therapy. Furthermore, either or both of coil electrodes 24 and 26 may be used in a cardiac pacing electrode vector for delivering cardiac pacing pulses according to the techniques disclosed herein. While two coil electrodes 24 and 26 are shown along lead body 18, in other examples only one coil electrode (which may be used in combination with housing 15 for delivering high voltage pulses) or three or more coil electrodes may be carried by lead body 18. In still other examples, two or more coil electrodes may be carried by two or more different lead bodies extending from ICD 14.

Electrodes 28 and 30 are relatively smaller surface area electrodes which are available for use in sensing electrode vectors for sensing cardiac electrical signals and may be used for delivering relatively low voltage cardiac pacing pulses in some examples. Electrodes 28 and 30 are sometimes referred to as “pace/sense electrodes” because they are generally configured for use in relatively low voltage applications, e.g., used as either a cathode or anode for delivery of pacing pulses and/or sensing of cardiac electrical signals, as opposed to delivering high voltage CV/DF shocks. In some instances, electrodes 28 and 30 may provide only pacing functionality, only sensing functionality or both.

Electrodes 28 and 30 may be ring electrodes extending around the circumference of lead body 18 and having a relatively short longitudinal dimension along the length of lead body 18 compared to coil electrodes 24 and 26. For the sake of convenience, electrodes 28 and 30 are referred to herein as “ring electrodes” to distinguish them from the relatively larger surface area, low impedance electrodes 24 and 26, referred to herein as “coil electrodes.” However, electrodes 28 and 30 may comprise any of a number of different types of electrodes, including ring electrodes, short coil electrodes, button electrodes, hemispherical electrodes, directional electrodes, segmented electrodes, helical electrodes, fishhook electrodes, tip electrodes, or the like and are not limited to being exclusively ring electrodes.

In the example illustrated in FIGS. 1A and 1B, ring electrode 28 is located proximal to coil electrode 24, and ring electrode 30 is located between coil electrodes 24 and 26. Ring electrodes 28 and 30 may be positioned at other locations along lead body 18 and are not limited to the positions shown. One, two or more ring or other low surface area electrodes used for sensing and/or low voltage cardiac pacing pulse delivery may be carried by lead body 18. For instance, a third ring electrode may be located distal to coil electrode 26 in some examples. In other examples, lead 16 may include fewer or more ring electrodes and/or coil electrodes than the example shown here.

In some cases, post-shock cardiac pacing pulses are needed to prevent asystole following a CV/DF shock until the intrinsic conduction system initiates an intrinsic heart rhythm. In other cases, cardiac pacing may be needed to treat bradycardia, asystole or deliver anti-tachycardia pacing (ATP), as examples. Cardiac pacing pulses are generally much lower in voltage than CV/DF shock pulses because a much smaller, relatively local volume of cardiac tissue can be captured by a pacing pulse to cause a heartbeat than the relatively large mass of cardiac tissue that is simultaneously depolarized during a CV/DF shock. Cardiac pacing pulses are delivered to cause depolarization of myocardial tissue at one or more local pacing sites. The pacing evoked depolarization of local cardiac cells captured in the vicinity of the pacing cathode electrode is conducted through the heart via the myocardium and/or intrinsic conduction system in a coordinated manner to cause a paced heartbeat.

In some pacemaker and ICD systems, cardiac pacing pulses can be delivered using relatively low surface area electrodes, similar to that of ring electrodes 28 and 30, carried by endocardial or epicardial leads so that the low surface area electrodes are in close or intimate contact with myocardial tissue. Pacing pulses delivered using low surface area, transvenous, endocardial electrodes, for example, may typically have a voltage amplitude up to a maximum of 8 volts (V) and a pulse width of 2.0 ms or less. More typically, a pacing pulse that successfully paces the heart via endocardial or epicardial electrodes might be 1.0 to 5.0 V, e.g., 2.5 V, in pulse amplitude with a 0.25 to 0.5 ms pulse width, as examples. The pulse amplitude and pulse width of the pacing pulse are selected to deliver sufficient energy to cause electrical depolarization of the myocardial tissue of the heart at the pacing site to thereby capture the heart and cause a heartbeat.

Cardiac pacing pulses that are delivered using extra-cardiac electrodes generally require higher energy (e.g., higher pulse amplitude and/or pulse width) than cardiac pacing pulses that are delivered using endocardial or epicardial electrodes but are still lower in pacing voltage amplitude and pulse energy than that required for CV/DF shocks. Relatively higher voltage cardiac pacing pulses are required when pacing using extracardiac electrodes in order to deliver enough energy within the pacing pulse width to capture the heart. A limitation of the maximum pacing pulse width may exist due in part to the decay rate of the pacing pulse amplitude which can be dependent on the capacitance of a capacitor being discharged to deliver the pacing pulse and the impedance of the pacing electrode vector. Therefore in order to achieve capture within a limited pulse width, e.g., 2 ms or less, a high pacing voltage amplitude may be required to deliver sufficient pacing pulse energy. Cardiac pacing pulses delivered using extra-cardiac electrodes may be in the range of 8 V to 40 V with a pacing pulse width of 2 ms to 8 ms, as examples. By comparison CV/DF shocks may be greater than 100 V or on the order of several hundred volts.

As described below, high surface area coil electrodes 24 and 26 may be employed for delivering cardiac pacing pulses. Relatively higher pacing pulse voltage amplitudes may be used with lower current density at the electrode tissue interface of the high surface area coil electrodes 24 and 26 compared to the low surface area electrodes 28 and 30. The surface area of a coil electrode 24 or 26 may be 50 to 100 times larger than the surface area of the ring electrodes 28 and 30. High current density at the ring electrode-tissue interface during relatively high voltage cardiac pacing could cause local tissue injury. The electrical field of current traveling through conductive tissues toward the heart between a cardiac pacing electrode vector that includes at least one or both high surface area coil electrodes 24 and 26 may be more effective in capturing the heart for cardiac pacing than the electrical field between a cardiac pacing electrode vector that includes lower surface area ring electrodes 28 and 30 or one of ring electrodes 28 or 30 and housing 15. A higher voltage cardiac pacing pulse that can be delivered via the coil electrodes 24 and 26 can have a relatively short pulse width so that the pacing pulse decay rate does not become a limiting factor of pacing pulse energy delivered for capturing the heart.

Accordingly, as described below, ICD 14 may be configured to deliver cardiac pacing pulses using coil electrodes 24 and 26, e.g., as a cathode and anode pair. High voltage output circuitry of ICD 14 is enabled by therapy delivery control circuitry of ICD 14 when a CV/DF shock is needed for delivery via coil electrodes 24 and/or 26. However, when a relatively high voltage cardiac pacing pulse is needed, that is much lower voltage than the CV/DF shock pulse, ICD 14 is configured to enable bypass circuitry for delivering a cardiac pacing pulse to one or both of coil electrodes 24 and 26 using only a portion of the high voltage output circuitry. Current required to operate the high voltage output circuitry is reduced by enabling the bypass circuitry for delivering a cardiac pacing pulse to the coil electrodes 24 and/or 26 via only a portion of the high voltage output circuitry compared to the current required to operate the high voltage output circuitry for delivering a CV/DF shock.

Referring again to the example shown in FIGS. 1A and 1B, lead 16 extends subcutaneously or submuscularly over the ribcage 32 medially from the connector assembly 27 of ICD 14 toward a center of the torso of patient 12, e.g., toward xiphoid process 20 of patient 12. At a location near xiphoid process 20, lead 16 bends or turns and extends superiorly, subcutaneously or submuscularly, over the ribcage and/or sternum, substantially parallel to sternum 22. Although illustrated in FIG. 1A as being offset laterally from and extending substantially parallel to sternum 22, the distal portion 25 of lead 16 may be implanted at other locations, such as over sternum 22, offset to the right or left of sternum 22, angled laterally from sternum 22 toward the left or the right, or the like. Alternatively, lead 16 may be placed along other subcutaneous or submuscular paths. The path of extra-cardiovascular lead 16 may depend on the location of ICD 14, the arrangement and position of electrodes carried by the lead body 18, and/or other factors. The techniques disclosed herein are not limited to a particular path of lead 16 or final locations of electrodes 24, 26, 28 and 30.

Electrical conductors (not illustrated) extend through one or more lumens of the elongated lead body 18 of lead 16 from the lead connector at the proximal lead end 27 to electrodes 24, 26, 28, and 30 located along the distal portion 25 of the lead body 18. The elongated electrical conductors contained within the lead body 18, which may be separate respective insulated conductors within the lead body 18, are each electrically coupled with respective coil electrodes 24 and 26 and ring electrodes 28 and 30. The respective conductors electrically couple the electrodes 24, 26, 28, and 30 to circuitry, such as a therapy delivery circuit and/or a sensing circuit, of ICD 14 via connections in the connector assembly 17, including associated electrical feedthroughs crossing housing 15. The electrical conductors transmit electrical stimulation pulses from a therapy delivery circuit within ICD 14 to one or more of coil electrodes 24 and 26 and/or ring electrodes 28 and 30 and transmit electrical signals produced by the patient's heart 8 from one or more of coil electrodes 24 and 26 and/or ring electrodes 28 and 30 to the sensing circuit within ICD 14.

The lead body 18 of lead 16 may be formed from a non-conductive material, including silicone, polyurethane, fluoropolymers, mixtures thereof, and/or other appropriate materials, and shaped to form one or more lumens within which the one or more conductors extend. Lead body 18 may be tubular or cylindrical in shape. In other examples, the distal portion 25 (or all of) the elongated lead body 18 may have a flat, ribbon or paddle shape. Lead body 18 may be formed having a preformed distal portion 25 that is generally straight, curving, bending, serpentine, undulating or zig-zagging.

In the example shown, lead body 18 includes a curving distal portion 25 having two “C” shaped curves, which together may resemble the Greek letter epsilon, “¿.” Defibrillation electrodes 24 and 26 are each carried by one of the two respective C-shaped portions of the lead body distal portion 25. The two C-shaped curves are seen to extend or curve in the same direction away from a central axis of lead body 18, along which ring electrodes 28 and 30 are positioned. Ring electrodes 28 and 30 may, in some instances, be approximately aligned with the central axis of the straight, proximal portion of lead body 18 such that mid-points of coil electrodes 24 and 26 are laterally offset from ring electrodes 28 and 30.

Other examples of extra-cardiovascular leads including one or more coil electrodes and one or more ring electrodes carried by curving, serpentine, undulating or zig-zagging distal portion of the lead body 18 that may be implemented with the techniques described herein are generally disclosed in U.S. Pat. No. 10,675,478 (Marshall, et al.). The techniques disclosed herein are not limited to any particular lead body design, however. In other examples, lead body 18 is a flexible elongated lead body without any pre-formed shape, bends or curves.

ICD 14 may obtain cardiac electrical signals corresponding to electrical activity of heart 8 via a combination of sensing electrode vectors that include combinations of electrodes 24, 26, 28 and/or 30. In some examples, housing 15 of ICD 14 is used in combination with one or more of electrodes 24, 26, 28 and/or 30 in at least one sensing electrode vector. Each cardiac electrical signal received via a selected sensing electrode vector may be used by ICD 14 for sensing cardiac event signals attendant to intrinsic depolarizations of the myocardium, e.g., R-waves attendant to ventricular depolarizations and in some cases P-waves attendant to atrial depolarizations. Sensed cardiac event signals may be used for determining the heart rate and determining a need for cardiac pacing, e.g., for treating bradycardia or asystole for preventing a long ventricular pause, or for determining a need for tachyarrhythmia therapy, e.g., ATP and/or CV/DF shocks.

ICD 14 analyzes the cardiac electrical signal(s) received from one or more sensing electrode vectors to monitor for abnormal rhythms, such as asystole, bradycardia, ventricular tachycardia (VT) and/or ventricular fibrillation (VF). ICD 14 may analyze the heart rate and/or morphology of the cardiac electrical signals to monitor for tachyarrhythmia in accordance with any tachyarrhythmia detection techniques. ICD 14 generates and delivers electrical stimulation therapy in response to detecting a tachyarrhythmia, e.g., VT or VF (VT/VF) using a therapy delivery electrode vector which may be selected from any of the available electrodes 24, 26, 28 30 and/or housing 15. ICD 14 may deliver ATP in response to VT detection and in some cases may deliver ATP prior to a CV/DF shock or during high voltage holding capacitor charging in an attempt to avert the need for delivering a CV/DF shock. If ATP does not successfully terminate VT or when VF is detected, ICD 14 may deliver one or more CV/DF shocks via one or both of coil electrodes 24 and 26 and/or housing 15.

In the absence of a ventricular event signal, e.g., a sensed R-wave, ICD 14 may generate and deliver a cardiac pacing pulse, such as a post-shock pacing pulse or bradycardia pacing pulse when asystole is detected or when a pacing escape interval expires prior to sensing a ventricular event signal, e.g., when AV block is present. The cardiac pacing pulses may be delivered using a pacing electrode vector that includes at least one or both coil electrodes 24 and 26 according to the techniques disclosed herein.

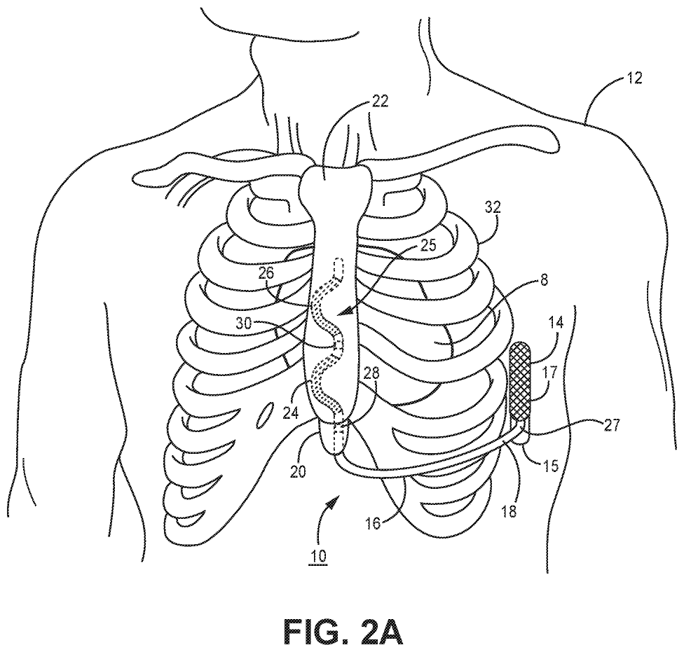

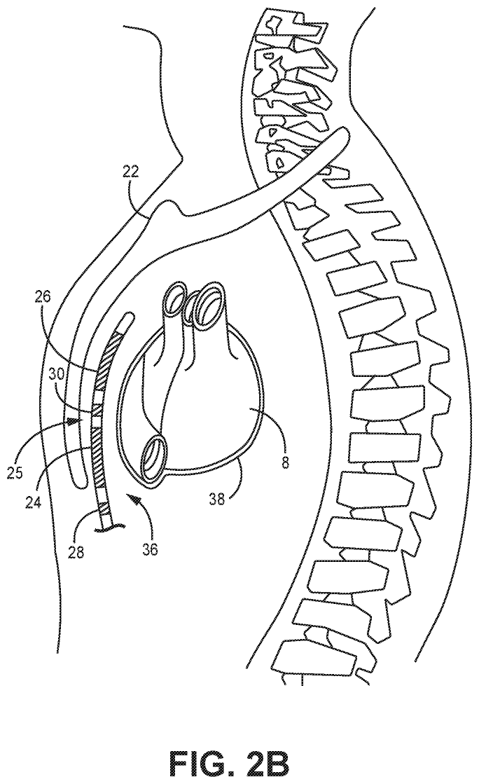

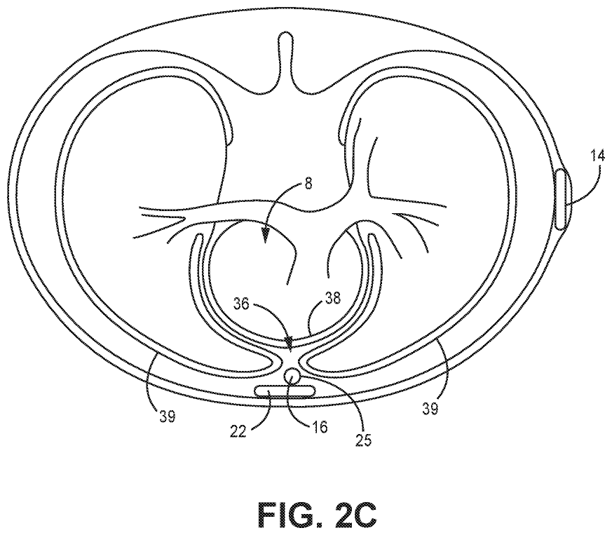

ICD 14 is shown implanted subcutaneously on the left side of patient 12 along the ribcage 32. ICD 14 may, in some instances, be implanted between the left posterior axillary line and the left anterior axillary line of patient 12. ICD 14 may, however, be implanted at other subcutaneous or submuscular locations in patient 12. For example, ICD 14 may be implanted in a subcutaneous pocket in the pectoral region. In this case, lead 16 may extend subcutaneously or submuscularly from ICD 14 toward the manubrium of sternum 22 and bend or turn and extend inferiorly from the manubrium to the desired location subcutaneously or submuscularly. In yet another example, ICD 14 may be placed abdominally. Lead 16 may be implanted in other extra-cardiovascular locations as well. For instance, as described with respect to FIGS. 2A-2C, the distal portion 25 of lead 16 may be implanted underneath the sternum/ribcage in the substernal space. FIGS. 1A and 1B are illustrative in nature and should not be considered limiting in the practice of the techniques disclosed herein.

A medical device operating according to techniques disclosed herein may be coupled to a transvenous or non-transvenous lead in various examples for carrying electrodes for sensing cardiac electrical signals and delivering electrical stimulation therapy. For example, the medical device, such as ICD 14, may be coupled to an extra-cardiovascular lead as illustrated in the accompanying drawings, referring to a lead that positions electrodes outside the blood vessels, heart, and pericardium surrounding the heart of a patient. Implantable electrodes carried by extra-cardiovascular leads may be positioned extra-thoracically (outside the ribcage and sternum), subcutaneously or submuscularly, or intra-thoracically (beneath the ribcage or sternum, sometimes referred to as a sub-sternal position) and may not necessarily be in intimate contact with myocardial tissue. An extra-cardiovascular lead may also be referred to as a “non-transvenous” lead.

In other examples, the medical device may be coupled to a transvenous lead that positions electrodes within a blood vessel, which may remain outside the heart in an extra-cardiac location or be advanced to position electrodes within a heart chamber. For instance, a transvenous medical lead may be advanced along a venous pathway to position electrodes in an extra-cardiac location within the internal thoracic vein (ITV), an intercostal vein, the superior epigastric vein, or the azygos, hemiazygos, or accessory hemiazygos veins, as examples. In still other examples, a transvenous lead may be advanced to position electrodes within the heart, e.g., within an atrial and/or ventricular heart chambers.

An external device 40 is shown in telemetric communication with ICD 14 by a wireless communication link 42 in FIG. 1A. External device 40 may include a processor 52, memory 53, display 54, user interface 56 and telemetry unit 58. Processor 52 controls external device operations and processes data and signals received from ICD 14. Display unit 54, which may include a graphical user interface, displays data and other information to a user for reviewing ICD operation and programmed parameters as well as cardiac electrical signals retrieved from ICD 14.

User interface 56 may include a mouse, touch screen, keypad or the like to enable a user to interact with external device 40 to initiate a telemetry session with ICD 14 for retrieving data from and/or transmitting data to ICD 14, including programmable parameters for controlling cardiac event signal sensing, arrhythmia detection and therapy delivery. Telemetry unit 58 includes a transceiver and antenna configured for bidirectional communication with a telemetry circuit included in ICD 14 and is configured to operate in conjunction with processor 52 for sending and receiving data relating to ICD functions via communication link 42.

Communication link 42 may be established between ICD 14 and external device 40 using a radio frequency (RF) link such as BLUETOOTH®, Wi-Fi, or Medical Implant Communication Service (MICS) or other RF or communication frequency bandwidth or communication protocols. Data stored or acquired by ICD 14, including physiological signals or associated data derived therefrom, results of device diagnostics, battery status, and histories of detected rhythm episodes and delivered therapies, etc., may be retrieved from ICD 14 by external device 40 following an interrogation command.

External device 40 may be embodied as a programmer used in a hospital, clinic or physician's office to retrieve data from ICD 14 and to program operating parameters and algorithms in ICD 14 for controlling ICD functions. External device 40 may alternatively be embodied as a home monitor or handheld device. External device 40 may be used to program cardiac signal sensing parameters, cardiac rhythm detection parameters and therapy control parameters used by ICD 14. At least some control parameters used in sensing cardiac event signals and detecting arrhythmias as well as therapy delivery control parameters may be programmed into ICD 14 using external device 40 in some examples. For example, a user may program a pacing voltage amplitude and pacing electrode vector that includes at least one or both coil electrodes 24 and 26. As described below, processing and control circuitry enclosed by housing 15 may select a cardiac pacing pulse voltage source and therapy delivery output circuitry for delivering cardiac pacing pulses via at least one coil electrode 24 or 26 based on the programmed pacing pulse voltage amplitude.

FIGS. 2A-2C are conceptual diagrams of patient 12 implanted with extra-cardiovascular ICD system 10 in a different implant configuration than the arrangement shown in FIGS. 1A-1B. FIG. 2A is a front view of patient 12 implanted with ICD system 10. FIG. 2B is a side view of patient 12 implanted with ICD system 10. FIG. 2C is a transverse view of patient 12 implanted with ICD system 10. In this arrangement, extra-cardiovascular lead 16 of system 10 is implanted at least partially underneath sternum 22 of patient 12. Lead 16 extends subcutaneously or submuscularly from ICD 14 toward xiphoid process 20 and at a location near xiphoid process 20 bends or turns and extends superiorly within anterior mediastinum 36 (see FIG. 2C) in a substernal position.

Anterior mediastinum 36 may be viewed as being bounded laterally by pleurae 39, posteriorly by pericardium 38, and anteriorly by sternum 22 (see FIG. 2C). The distal portion 25 of lead 16 may extend along the posterior side of sternum 22 substantially within the loose connective tissue and/or substernal musculature of anterior mediastinum 36. A lead implanted such that the distal portion 25 is substantially within anterior mediastinum 36, may be referred to as a “substernal lead.”

In the example illustrated in FIGS. 2A-2C, lead 16 is located substantially centered under sternum 22. In other instances, however, lead 16 may be implanted such that it is offset laterally from the center of sternum 22. In some instances, lead 16 may extend laterally such that distal portion 25 of lead 16 is underneath/below the ribcage 32 in addition to or instead of sternum 22. In other examples, the distal portion 25 of lead 16 may be implanted in other extra-cardiac, intra-thoracic locations, including in the pleural cavity or around the perimeter of and adjacent to the pericardium 38 of heart 8.

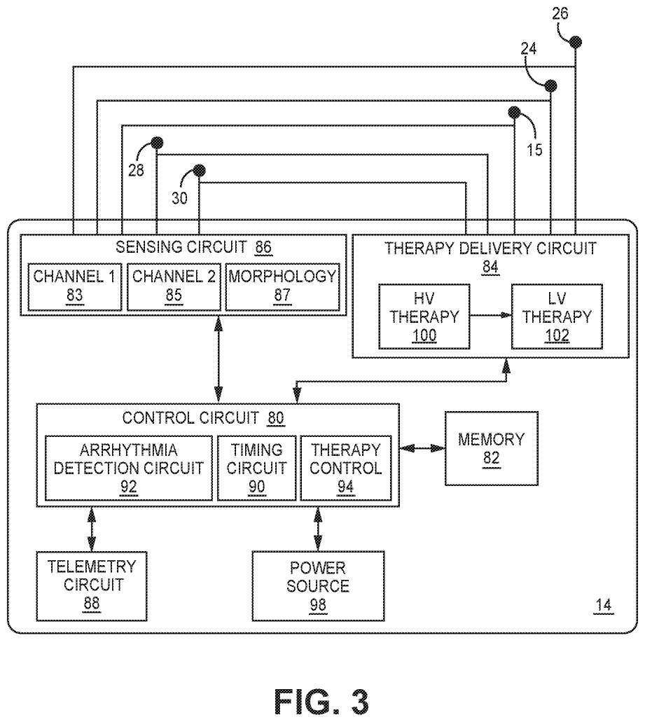

FIG. 3 is a conceptual diagram of ICD 14 according to one example. The electronic circuitry enclosed within housing 15 (shown schematically as an electrode, sometimes referred to as a “can electrode,” in FIG. 3) includes software, firmware and hardware that cooperatively monitor cardiac electrical signals, determine when an electrical stimulation therapy is necessary, and deliver therapy as needed according to programmed therapy delivery algorithms and control parameters. ICD 14 may be coupled to a lead, such as lead 16 carrying electrodes 24, 26, 28, and 30 as shown in the examples of FIGS. 1A-2C, for delivering electrical stimulation pulses to the patient's heart and for sensing cardiac electrical signals.

ICD 14 includes a control circuit 80, memory 82, therapy delivery circuit 84, cardiac electrical signal sensing circuit 86, and telemetry circuit 88. A power source 98 provides power to the circuitry of ICD 14, including each of the components 80, 82, 84, 86, and 88 as needed. Power source 98 may include one or more energy storage devices, such as one or more rechargeable or non-rechargeable batteries. The connections between power source 98 and each of the other components 80, 82, 84, 86 and 88 are to be understood from the general block diagram of FIG. 3 but are not shown for the sake of clarity. For example, power source 98 may be coupled to one or more charging circuits included in therapy delivery circuit 84 for charging holding capacitors included in therapy delivery circuit 84 and operating output circuitry for discharging the holding capacitor(s) at appropriate times under the control of control circuit 80 for producing electrical pulses according to a therapy protocol. Power source 98 is also coupled to components of cardiac electrical signal sensing circuit 86 (such as sense amplifiers, analog-to-digital converters, switching circuitry, etc.), memory 82, and telemetry circuit 88 as needed.

The operating circuits shown in FIG. 3 represent functionality included in ICD 14 and may include any discrete and/or integrated electronic circuit components that implement analog and/or digital circuits capable of producing the functions attributed to ICD 14 herein. Functionality associated with one or more circuits may be performed by separate hardware, firmware and/or software components, or integrated within common hardware, firmware and/or software components. For example, cardiac electrical signal sensing and analysis for detecting arrhythmia may be performed cooperatively by sensing circuit 86 and control circuit 80 and may include operations implemented in a processor or other signal processing circuitry included in control circuit 80 executing instructions stored in memory 82 and control signals such as blanking and timing intervals and sensing threshold amplitude signals sent from control circuit 80 to sensing circuit 86. Therapy delivery may be performed cooperatively by therapy delivery circuit 84 under the control of signals received from control circuit 80 for controlling the timing, amplitude, width, polarity, rate, electrode vector and other therapy delivery parameters used by therapy delivery circuit to generate and deliver electrical stimulation pulses, which may include CV/DF pulses, cardiac pacing pulses, tachyarrhythmia induction pulses, impedance measurement pulses or any other electrical pulses delivered via electrodes 24, 26, 28, 30 and/or housing 15.

The various circuits of ICD 14 may include an application specific integrated circuit (ASIC), an electronic circuit, a processor (shared, dedicated, or group) and memory that execute one or more software or firmware programs, a combinational logic circuit, state machine, hardware subroutine, or other suitable components or combinations of components that provide the described functionality. The particular form of software, hardware and/or firmware employed to implement the functionality disclosed herein will be determined primarily by the particular system architecture employed in the ICD and by the particular sensing, detection and therapy delivery methodologies employed by the ICD. Providing software, hardware, and/or firmware to accomplish the described functionality in the context of any modern medical device system, given the disclosure herein, is within the abilities of one of skill in the art.

Memory 82 may include any volatile, non-volatile, magnetic, or electrical non-transitory computer readable storage media, such as random access memory (RAM), read-only memory (ROM), non-volatile RAM (NVRAM), electrically-erasable programmable ROM (EEPROM), flash memory, or any other memory device. Furthermore, memory 82 may include non-transitory computer readable media storing instructions that, when executed by one or more processing circuits, cause control circuit 80 and/or other ICD components to perform various functions attributed to ICD 14 or those ICD components. The non-transitory computer-readable media storing the instructions may include any of the media listed above.

Therapy delivery circuit 84 and sensing circuit 86 are electrically coupled to electrodes 24, 26, 28, 30 carried by lead 16 and the housing 15, which may function as a common or ground electrode for sensing or cardiac pacing pulses or as an active can electrode for delivering CV/DF shock pulses. Control circuit 80 communicates, e.g., via a data bus, with therapy delivery circuit 84 and sensing circuit 86 for sensing cardiac electrical signals, detecting cardiac rhythms, and controlling delivery of cardiac electrical stimulation therapies in response to sensed cardiac signals (or the absence thereof). Control circuit 80 may include an arrhythmia detection circuit 92, timing circuit 90, and therapy control circuit 94. Arrhythmia detection circuit 92 may be configured to process and analyze signals received from sensing circuit 86, which may be in conjunction with time intervals and/or timing related signals received from timing circuit 90. Timing circuit 90 may generate clock signals and include various timers and/or counters for use in determining time intervals between cardiac events, sensed and/or paced, and control the timing of delivered pacing pulses and/or CV shocks. Control circuit 80 may further include a therapy control circuit 94 configured to pass signals to and receive signals from therapy delivery circuit 84 for controlling and monitoring electrical stimulation therapies delivered by therapy delivery circuit 84.

Cardiac electrical signal sensing circuit 86 (also referred to herein as “sensing circuit” 86) may be selectively coupled to electrodes 28, 30 and/or housing 15 in order to monitor electrical activity of the patient's heart. Sensing circuit 86 may additionally be selectively coupled to defibrillation electrodes 24 and/or 26 for use in a sensing electrode vector together or in combination with one or more of electrodes 28, 30 and/or housing 15. Sensing circuit 86 may be enabled to receive cardiac electrical signals from at least one sensing electrode vector selected from the available electrodes 24, 26, 28, 30, and housing 15 in some examples. At least two, three or more cardiac electrical signals from two, three or more different sensing electrode vectors may be received simultaneously by sensing circuit 86 in some examples. Sensing circuit 86 may monitor one or more cardiac electrical signals for sensing cardiac event signals, e.g., R-waves attendant to intrinsic ventricular myocardial depolarizations. In some examples, sensing circuit 86 may be configured to monitor two cardiac electrical signals simultaneously for sensing cardiac event signals. At least one cardiac electrical signal may be received by sensing circuit 86 and passed to control circuit 80 for processing and analysis, e.g., by arrhythmia detection circuit 92, for determining when morphology-based criteria for detecting arrhythmia are met in some examples.

In the example shown, sensing circuit 86 may include switching circuitry for selecting which of electrodes 24, 26, 28, 30, and housing 15 are coupled as a first sensing electrode vector to a first sensing channel 83 for receiving a first cardiac electrical signal, which electrodes are coupled as a second sensing electrode vector to a second sensing channel 85 of sensing circuit 86 for receiving a second cardiac electrical signal, and which electrodes are coupled as a third sensing electrode vector to a morphology signal channel 87 for receiving a third cardiac electrical signal.

Each sensing channel 83 and 85, when included, may be configured to amplify, filter and digitize the cardiac electrical signal received from selected electrodes coupled to the respective sensing channel to improve the signal quality for sensing cardiac event signals, such as R-waves. The cardiac event detection circuitry within sensing circuit 86 may include one or more sense amplifiers, filters, rectifiers, threshold detectors, comparators, analog-to-digital converters (ADCs), timers or other analog and/or digital components. A cardiac event sensing threshold may be automatically adjusted by each sensing channel 83 and 85 under the control of control circuit 80, based on sensing threshold control parameters, such as various timing intervals and sensing threshold amplitude values that may be determined by control circuit 80, stored in memory 82, and/or controlled by hardware, firmware and/or software of control circuit 80 and/or sensing circuit 86. In response to sensing a cardiac event signal, e.g., an R-wave, sensing circuit 86 may generate a sensed event signal, e.g., a ventricular sensed event signal, that is passed to control circuit 80.

Ventricular sensed event signals received from sensing circuit 86 by control circuit 80 can be used by control circuit 80 for determining sensed event intervals, which can be referred to as RR intervals (RRIs). An RRI is the time interval between two ventricular sensed event signals received by control circuit 80. Control circuit 80 may include a timing circuit 90 for determining RRIs. Based on RRIs, control circuit 80 may detect VT/VF in some examples. RRIs may include time intervals between consecutive ventricular sensed event signals and intervals between a delivered pacing pulse and a ventricular sensed event signal.

In some examples, sensing circuit 86 receives a third cardiac electrical signal by morphology signal channel 87 for passing a digitized electrocardiogram (ECG) signal to control circuit 80 for morphology analysis. The three cardiac electrical signals sensed by sensing circuit 86 may be received using three different sensing electrode vectors selected from the available electrodes 24, 26, 28 and 30 and housing 15. In other examples, two cardiac electrical signals may be received by sensing circuit 86 from two different sensing electrode vectors, with one signal passed to the first sensing channel 83 and the other signal passed to the second sensing channel 85. Either or both of the two signals may be passed to control circuit 80 as a multi-bit digital ECG signal used by control circuit 80 for morphology analysis of the cardiac signal. Multiple channels 83, 85 and 87 may be optional in some examples, however. Aspects of the techniques disclosed herein for delivering therapeutic electrical stimulation pulses may be implemented in conjunction with a variety of cardiac event signal sensing and arrhythmia detection methods and are not limited to any particular method for determining the need or timing of an electrical pulse delivered by therapy delivery circuit 84.

Timing circuit 90 may be configured to control various timers and/or counters used in setting various intervals and windows used in sensing ventricular event signals, determining time intervals between received ventricular sensed event signals, performing morphology analysis and controlling the timing of cardiac pacing pulses generated by therapy delivery circuit 84. Timing circuit 90 may start a timer in response to receiving ventricular sensed event signals from sensing channels 83 and 85 and for timing RRIs. Timing circuit 90 may pass the RRIs to arrhythmia detection circuit 92 for determining and counting tachyarrhythmia intervals.

Control circuit 80 may include an arrhythmia detection circuit 92 configured to analyze RRIs received from timing circuit 90 and cardiac electrical signals received from morphology signal channel 87 for detecting arrhythmia. Arrhythmia detection circuit 92 may be configured to detect asystole and/or tachyarrhythmia based on sensed cardiac electrical signals meeting respective asystole or tachyarrhythmia detection criteria. Arrhythmia detection circuit 92 may be implemented in control circuit 80 as hardware, software and/or firmware that processes and analyzes signals received from sensing circuit 86 for detecting VT/VF. In some examples, arrhythmia detection circuit 92 may include comparators and counters for counting RRIs determined by timing circuit 90 that are tachyarrhythmia intervals. An RRI that is less than the tachyarrhythmia detection interval is referred to as a “tachyarrhythmia interval.” Arrhythmia detection circuit 92 may compare the RRIs determined by timing circuit 90 to one or more tachyarrhythmia detection interval zones, such as a VT detection interval zone and a VF detection interval zone. RRIs falling into a detection interval zone are counted by a respective VT interval counter or VF interval counter and in some cases in a combined VT/VF interval counter. When a threshold number of tachyarrhythmia intervals is reached, control circuit 80 may detect VT or VF. In some examples, a tachyarrhythmia detection based on the threshold number of tachyarrhythmia intervals being reached may be confirmed or rejected based on morphology analysis of a cardiac electrical signal.

The VF detection interval threshold may be set to 280 to 350 milliseconds (ms), as examples. When VT detection is enabled, the VT detection interval may be programmed to be in the range of 350 to 420 ms, or 400 ms as an example. VT or VF may be detected when the respective VT or VF interval counter (or a combined VT/VF interval counter) reaches a threshold number of intervals to detect (NID). As an example, the NID to detect VT may require that the VT interval counter reaches 18, 24, 32 or other selected number of VT intervals. The VT intervals may or may not be required to be consecutive intervals. The NID required to detect VF may be programmed to a threshold number of X VF intervals out of Y consecutive RRIs. For instance, the NID required to detect VF may be 18 VF intervals out of the most recent 24 consecutive RRIs, 30 VF intervals out 40 consecutive RRIs, or as high as 120 VF intervals out of 160 consecutive RRIs as examples.

Arrhythmia detection circuit 92 may be configured to perform other signal analysis for determining if other detection criteria are satisfied before detecting VT or VF based on an NID being reached, such as R-wave morphology criteria, onset criteria, stability criteria and noise and oversensing rejection criteria. To support these additional analyses, sensing circuit 86 may pass a digitized ECG signal to control circuit 80, e.g., from morphology signal channel 87, for morphology analysis performed by arrhythmia detection circuit 92 for detecting and discriminating heart rhythms. A cardiac electrical signal received by the morphology signal channel 87 (and/or sensing channel 83 and/or sensing channel 85) may be passed through a filter and amplifier, provided to a multiplexer and thereafter converted to a multi-bit digital signal by an analog-to-digital converter, all included in sensing circuit 86, for storage in memory 82. Memory 82 may include one or more circulating buffers to temporarily store digital cardiac signal segments for analysis performed by control circuit 80. Control circuit 80 may be a microprocessor-based controller that employs digital signal analysis techniques to characterize the digitized signals stored in memory 82 to recognize and classify the patient's heart rhythm employing any of numerous signal processing methodologies for analyzing cardiac signals and cardiac event waveforms, e.g., R-waves.

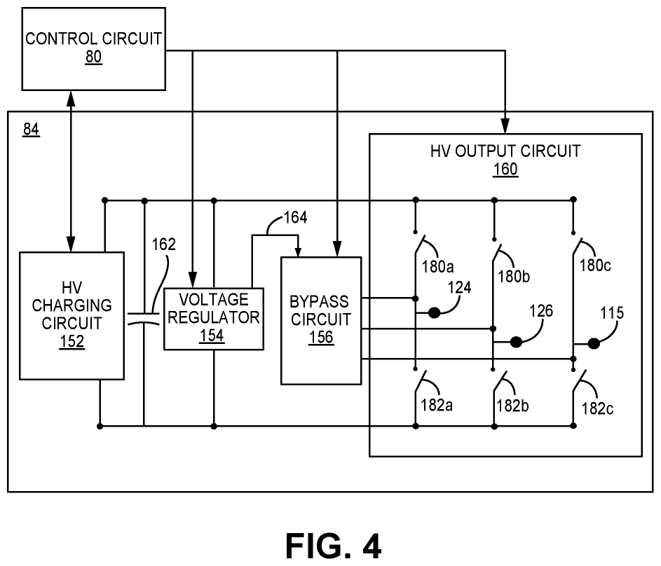

Therapy delivery circuit 84, as described below in conjunction with FIG. 4, may include at least one charging circuit and one or more charge storage devices such as one or more high voltage capacitors for generating high voltage shock pulses for treating VT/VF. Therapy delivery circuit 84 may include a high voltage (HV) therapy circuit 100, which may include a HV charging circuit, HV holding capacitor(s), and HV output circuit that are operatively controlled by signals from control circuit 80 for charging and subsequently discharging the high voltage capacitor(s) for CV/DF shock delivery when control circuit 80 detects VT/VF.

In some examples, therapy delivery circuit 84 may include a low voltage (LV) therapy delivery circuit 102, which may include a LV charging circuit, one or more LV holding capacitors and a LV output circuit for generating and delivering low voltage cardiac pacing pulses, e.g., cardiac pacing pulses having a pacing pulse amplitude that is 8 V or less, up to 10 V, up to 12 V, up to 16 V, or other maximum voltage amplitude of the LV therapy delivery circuit 102. LV cardiac pacing pulses may be delivered via ring electrodes 28 and/or 30 (together or in combination with housing 15) in some instances for successfully capturing and pacing the heart. Composite cardiac pacing pulses may be delivered by LV therapy delivery circuit 102 in some examples for delivering successive low voltage cardiac pacing pulses having a relatively long cumulative pulse width, e.g., up to 6 to 8 ms as examples, for delivering sufficient pulse energy to capture and pace the heart. Methods and devices for delivering composite cardiac pacing pulses, sometimes referred to as “stacked pacing pulses,” are generally disclosed in U.S. Pat. No. 10,449,362 (Anderson, et al.).

In some patients, however, the cardiac pacing capture threshold may require a pacing pulse amplitude and/or pulse width that is greater than a maximum pacing pulse amplitude and/or pulse width that can be generated and delivered by the LV therapy delivery circuit 102 via ring electrodes 28 and 30 for successfully capturing the heart. The pacing capture threshold and/or other factors, such as the electrical field of the pacing electrode vector relative to the patient's heart, current density at the electrode tissue interface, or auxiliary capture of non-cardiac tissue may make cardiac pacing via coil electrode 24 and/or coil electrode 26 desirable or preferred.

Cardiac pacing pulses using the high surface area coil electrodes 24 and 26 that are used to deliver CV/DF shock pulses may successfully capture the heart without limitations that may be associated with delivering cardiac pacing pulses from the LV therapy circuit 102 via the relatively small surface area ring electrodes 28 and 30 implanted at an extra-cardiac location. Delivery of cardiac pacing pulses by the HV therapy circuit 100, however, may prematurely drain current from power source 98. As further described below in conjunction with FIG. 4, HV output circuitry included in HV therapy circuit 100 may include switches and/or other components that require a relatively high operating current for enabling delivery of a CV/DF shock. CV/DF shocks are generally delivered relatively infrequently such that the current required to operate HV output circuitry may be acceptable over the usable life of ICD 14. However, a higher number of cardiac pacing pulses and/or more frequent cardiac pacing may be required over the life of the ICD 14 than CV/DF shocks, e.g., for delivering bradycardia pacing, ATP, post-shock pacing, etc. As such, the relatively high current required to operate the HV output circuitry of HV therapy circuit 100 for delivering cardiac pacing pulses via coil electrodes 24 and 26 may unacceptably shorten the useful life of power source 98.

Accordingly, therapy delivery circuit 84 may include operative circuitry referred to herein as a “bypass circuit” for conducting a pacing pulse signal from a cardiac pacing voltage source to coil electrode terminals while bypassing at least a portion of the HV output circuitry components that require a relatively high operating current for delivering CV/DF shock pulses to coil electrode terminals. In some examples, as described below in conjunction with FIG. 4, therapy delivery circuit 84 includes a voltage regulator configured to step down and/or hold the voltage amplitude of the charged HV capacitor(s) for generating cardiac pacing pulses having a pacing voltage amplitude that is relatively low in voltage amplitude compared to the CV/DF shocks delivered by HV therapy delivery circuit 84 but can be higher in voltage amplitude than the cardiac pacing pulses generated by LV therapy circuit 102. The voltage regulator used as a cardiac pacing voltage source and the bypass circuit may be controlled by therapy control circuit 94 to deliver cardiac pacing pulses via coil electrodes 24 and/or 26 having a voltage amplitude that is intermediate to the voltage amplitude of CV/DF shocks generated and delivered by HV therapy circuit 100 and the cardiac pacing pulses generated and delivered by the LV therapy delivery circuit 102.

As further described below, the bypass circuit may be controlled by therapy control circuit 94 for coupling a cardiac pacing voltage source to the coil electrodes 24 and/or 26 for delivering cardiac pacing using a portion of the HV output circuit while bypassing or excluding at least one high current component of the HV output circuit to reduce the operating current required to deliver a cardiac pacing pulse via coil electrode 24 and/or coil electrode 26 compared to delivering the cardiac pacing pulse via the HV output circuit according to the CV/DF output pathway. In some examples, the bypass circuit may be configured to receive an output voltage signal from the LV therapy delivery circuit 102 for delivering cardiac pacing pulses via the coil electrodes 24 and/or 26 using a cardiac pacing pathway that includes a portion of the HV output circuit but excludes high operating current components of the HV output circuit. The therapy control circuit 94 of control circuit 80 may select a cardiac pacing voltage source, the pacing voltage amplitude, pulse width, polarity and other characteristics of cardiac pacing pulses, which may be based on programmed values stored in memory 82.

In some examples, in addition to being configured to deliver therapeutic electrical stimulation pulses to the patient's heart under the control circuit 80, therapy delivery circuit 84 may be controlled to deliver electrical stimulation pulses for inducing tachyarrhythmia, e.g., T-wave shocks or trains of induction pulses, upon receipt of a programming command from external device 40 (FIG. 1A) by telemetry circuit 88, e.g., during ICD implant or follow-up testing procedures.

Telemetry circuit 88 includes a transceiver and antenna for communicating with external device 40 (shown in FIG. 1A) using RF communication or other communication protocols as described above. Control parameters utilized by control circuit 80 for sensing cardiac event signals, detecting arrhythmias, and controlling therapy delivery may be programmed into memory 82 via telemetry circuit 88. Under the control of control circuit 80, telemetry circuit 88 may receive downlink telemetry from and send uplink telemetry to external device 40. Telemetry circuit 88 may receive a pacing voltage amplitude, for example, selected and programmed by a user interacting with external device 40. Therapy control circuit 94 may select the cardiac pacing voltage source and pacing output pathway in accordance with the pacing voltage amplitude and pass control signals to therapy delivery circuit 84 for controlling delivery of pacing pulses by therapy delivery circuit 84 according to the selected pacing parameters.

FIG. 4 is a conceptual diagram of circuitry that can be included in therapy delivery circuit 84 of ICD 14 according to some examples. Therapy delivery circuit 84 includes HV charging circuit 152 configured to charge one or more HV holding capacitors 162 to deliver CV/DF shocks to coil electrodes 24 and/or 26 and/or housing 15 via HV output circuit 160. HV charging circuit 152, HV holding capacitor 162, and HV output circuit 160 may be included in the HV therapy circuit 100 shown in FIG. 3.

In response to control circuit 80 detecting a need for CV/DF shock therapy based on an analysis of cardiac electrical signals sensed by sensing circuit 86, HV holding capacitor 162 may be charged to a shock voltage amplitude by HV charging circuit 152 for delivering a CV/DF shock under the control of control circuit 80. HV charging circuit 152 may include a transformer to step up the battery voltage of power source 98 (shown in FIG. 3) in order to achieve charging of HV holding capacitor 162 to a voltage greater than the battery voltage. HV charging circuit 152 may include one or more transformers, switches, diodes, and/or other devices for operating to charge HV holding capacitor 162 to a desired voltage.

Control circuit 80 may pass a charge signal to HV charging circuit 152 to initiate charging and receive feedback signals from the HV charging circuit 152 to determine when HV holding capacitor 162 is charged to a shock voltage amplitude, e.g., corresponding to a programmed CV/DF shock energy, which may be selected based on defibrillation threshold testing or set to a nominal defibrillation energy, e.g., 20 Joules or more. A charge completion signal may be passed from control circuit 80 to HV charging circuit 152 to terminate charging of HV holding capacitor 162 in response to determining that the HV holding capacitor 162 is charged to a desired voltage.

While HV holding capacitor 162 is illustrated as a single capacitor in FIG. 4, it is to be understood that a combination of capacitors may be configured to function as a HV holding capacitor chargeable to a shock voltage amplitude. For example, two or more HV capacitors may be provided in HV therapy circuit 100 having an effective capacitance of 100 to 200 microfarads, or about 140 to 160 microfarads as examples. The HV capacitors may be charged to hold 750 to 800 V, for example, in order to deliver CV/DF shocks having a pulse energy of 5 Joules or more, and more typically 20 Joules or more.

A CV/DF shock can be delivered to the heart by discharging HV holding capacitor 162 under the control of control circuit 80 according to signals passed to HV output circuit 160, e.g., via a control bus from therapy control circuit 94. HV output circuit 160 includes switching circuitry, which may be in the form of an H-bridge including high side switches 180a-180c and low side switches 182a-182c, that are biased into a conducting state (e.g., on or enabled) from a non-conducting state (e.g., off or disabled) by signals from therapy control circuit 94 of control circuit 80.

High side switches 180a-180c may each include one or more electronic switching devices. In some examples, high side switches 180a-180c may each include an anode gated thyristor (AGT), metal oxide semiconductor field effect transistor (MOSFET), insulated gate bipolar transistor (IGBT), MOS-controlled thyristor (MCT), silicon-controlled rectifier (SCR) or other switching device or combination of switching devices having a high voltage rating. The high side switches 180a-c are generally high voltage rated switches that require a high operating current to bias the switch into a conducting or on state from a non-conducting or off state such that current leakage from HV holding capacitor 162 can be minimized when high side switches 180a-c are not enabled. High side switches 180a-180c may be charge coupled devices, such as AGTs, that can be controlled without requiring bootstrapping. One or a combination of high side switches 180a-180c is/are switched on and held in a conducting state for conducting current from the HV capacitor 162 to an electrode terminal 124, 126, or 115 coupled to coil electrode 24, coil electrode 26, or housing 15, respectively, selected as the CV/DF cathode electrode. A different one of coil electrode 26, coil electrode 24 or housing 15 may be selected as the return anode electrode by switching on a selected one of low side switches 182a, 182b or 182c, which is coupled to the respective electrode terminal 124, 126, 115 of the selected anode electrode.

A relatively high current trigger signal may be passed from control circuit 80 to switch on a selected high side switch 180a, 180b or 180c to start discharging HV capacitor 162 for shock delivery. During discharging of HV capacitor 162 through a selected shock delivery pathway, the high current flowing through the enabled high side switch 180a, 180b or 180c holds the switch in the conducting state until the switch 180a, 180b or 180c is disabled or switched “off” by control circuit 80. High side switches 180a-c may require a relatively high trigger current from control circuit 80 of 100 to 200 milliamps, for example, to bias the switch into a conducting state.

Low side switches 182a-182c may each include one or more switching devices, which may be implemented as SCRs, IGBTs, MOSFETs, MCTs, and/or other components or combinations of components. A low side switch 182a, 182b or 182c is biased in a conducting state by a control signal from therapy control circuit 94 of control circuit 80 to select a return path through an anode electrode selected from coil electrodes 24 and/or 26 or housing 15. Low side switches 182a-182c can be relatively low impedance switches, to minimize losses during defibrillation, and can be switched on by a relatively low current control signal, e.g., less than 10 milliamps, from control circuit 80.

Switches 180a-180c and switches 182a-182c are controlled to be on or off by control circuit 80 (e.g., by signals received from therapy control circuit 94 shown in FIG. 3) at the appropriate times for delivering a CV/DF shock. For instance, one of switches 180a, 180b or 180c may be switched on simultaneously with one of switches 182a, 182b, or 182c, without switching on both of the “a,” “b” or “c” switches across a given electrode terminal 124, 126 or 115, respectively, at the same time. To deliver a biphasic CV/DF shock using coil electrode 24 and housing 15, for instance, switch 180a and 182c may be switched on to deliver a first phase of the biphasic shock pulse. Before HV capacitor 162 is fully discharged, switches 180a and 182c are switched off after the first phase, and switches 180c and 182a are switched on to deliver the second phase of the biphasic pulse. Switches 180b and 182b remain off or in a non-conducting state in this example when coil electrode 26 is not selected for use in the CV/DF shock delivery vector. In other examples, coil electrode 26 may be included instead of coil electrode 24 or simultaneously selected with coil electrode 24 to function as a cathode electrode or an anode electrode. Examples of circuitry and techniques for delivering a CV/DF shock pulse via HV output circuitry are generally disclosed in U.S. Pat. No. 10,159,847 (Rasmussen, et al.).

When a cardiac pacing pulse is needed and the pacing capture threshold is very high, e.g., greater than 16 V, greater than 20 V, greater than 30 V or greater than 40 V, control circuit 80 may control HV charging circuit 152 to charge HV capacitor 162 to a programmed pacing voltage amplitude, less than the voltage required for CV/DF shock delivery. A relatively high voltage cardiac pacing pulse may be delivered via HV output circuit 160 by applying control signals to selected high side switches 180a-c and selected low side switches 182a-c as needed for discharging HV capacitor 162 via a selected pacing electrode vector including coil electrodes 24 and/or 26 and/or housing 15. However, current required to bias the selected switches 180a, 180b and/or 180c into a conducting state from a non-conducting state and the cardiac pacing pulses delivered using a high enough voltage amplitude for holding selected high side switches 180a, 180b and/or 180c in a conducting state during a cardiac pacing pulse can drain power source 98 prematurely. The relatively high voltage pacing pulses may not be well tolerated by the patient. As such, whenever possible (e.g., when the capture threshold falls in an intermediate or relatively lower voltage amplitude range), control circuit 80 may select a cardiac pacing voltage source of therapy delivery circuit 84 for delivering cardiac pacing via a cardiac pacing output pathway that is more power efficient than the output path that includes high side switches 180a-c of HV output circuit 160.

In some instances, as further described below, in response to control circuit 80 detecting a need for cardiac pacing, HV holding capacitor 162 may be charged to a voltage that is less than the voltage required for delivering a CV/DF shock for generating a rail voltage that is regulated by voltage regulator 154 for providing a pacing voltage source for delivering cardiac pacing pulses via a portion of HV output circuit 160. When the pacing capture threshold and corresponding pacing voltage amplitude is higher than a maximum voltage output that can be generated by LV therapy circuit 102, the HV holding capacitor 162 may be charged to a voltage amplitude used for generating a cardiac pacing pulse under the control of control circuit 80.

Bypass circuit 156 can be controlled by control circuit 80 to pass current from a cardiac pacing voltage source to electrode terminals 124, 126 and/or 115, respectively coupled to coil electrodes 124 and 126 and housing 115. When bypass circuit 156 is enabled by control circuit 80, the high side switches 180a-180c can be disabled by control signals from control circuit 80 for selecting a cardiac pacing output pathway that excludes a first portion of HV output circuit 160 that requires relatively high operating current and includes a second portion of HV output circuit 160 that requires relatively lower operating current. The first portion may include the high side switches 180a-c used during CV/DF shock delivery. The second portion may include any of the low side switches 182a, 182b and/or 182c, which require relatively lower operating current than the high side switches 180a-c and provide a return current path during pacing pulse delivery.

In some examples, therapy delivery circuit 84 may include cardiac pacing voltage source that includes voltage regulator 154 configured to pass a voltage output signal 164 to the bypass circuit 156 for delivering the cardiac pacing pulse to coil electrodes 24 and/or 26 without switching on any of high side switches 180a-c of HV output circuit 160. Switches 180a-c, which require a high operating current, are bypassed in a pacing output pathway when control circuit 80 enables bypass circuit 156.

Charging of HV capacitor 162 by HV charging circuit 152 may be controlled by control circuit 80 to produce a rail voltage, e.g., 10 to 50 V or about 20 to 40 V as examples, for providing a positive DC voltage that can be used to power various components of therapy delivery circuit 84. Voltage regulator 154 may receive the rail voltage and provide a voltage regulated output signal 164 having a desired cardiac pacing pulse voltage amplitude, which may be stepped down from the rail voltage, to bypass circuit 156. For example, HV charging circuit 152 may be controlled by control circuit 80 to charge the HV capacitor 162 to 16 V, 18 V, 20 V, 30 V, 40 V, 50 V or higher to generate a rail voltage that is at least equal to or greater than a desired cardiac pacing pulse voltage amplitude. Voltage regulator 154 may be configured to regulate the rail voltage to a programmed pacing pulse voltage amplitude, e.g., 15 to 30 V or about 16 to 20 V as examples. In some examples, voltage regulator 154 may be configured to set the voltage amplitude of output signal 164 to a fixed value, e.g., 16 to 18 V. In other examples, voltage regulator 154 may receive a control signal from control circuit 80 for adjusting the amplitude of the output signal 164 to a programmed pacing pulse voltage amplitude.

Bypass circuit 156 may include switching circuitry that can be enabled by a control signal from control circuit 80 and held in a conducting state by current flowing from voltage regulator 154, through bypass circuit 156, to a selected cardiac pacing cathode terminal 124, 126 and/or 115, and returning via a selected cardiac pacing anode terminal (a different one of terminals 124, 126 or 115 that is not selected as the cathode). The return anode may be selected by control circuit 80 via a control signal that turns on one of low side switches 182a-c of HV output circuit 160. Therapy control circuit 94 of control circuit 80 may control the pacing pulse polarity and pacing pulse width according to control signals passed to the switching devices of bypass circuit 156 and/or switches 182a-182c.

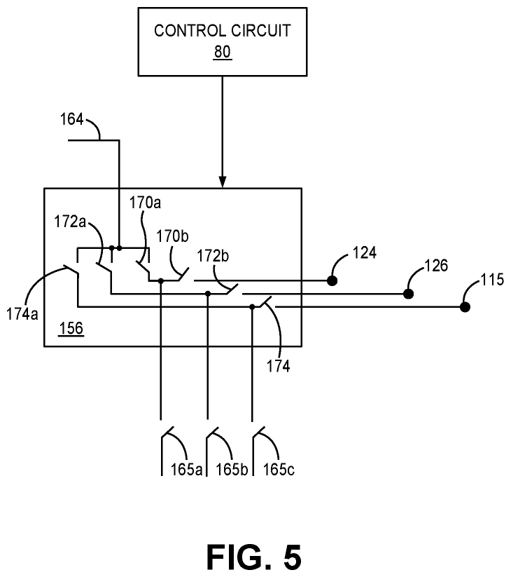

Bypass circuit 156 may include multiple switches, e.g., FETs or other solid state semiconductor devices, that require a relatively lower operating current to switch on and maintain in a conducting state than the current required to operate HV output circuit high side switches 180a-c. As described below in conjunction with FIG. 5, bypass circuit 156 may include multiple “channels,” with each channel including one or more switching devices for selectively coupling the voltage output signal 164, which is also referred to herein as a “cardiac pacing pulse signal,” to a desired pacing cathode electrode terminal from among terminals 124, 126 and 115. Control circuit 80 may control the switches of bypass circuit 156 and the low side switches 182a-c of HV output circuit 160 to deliver a monophasic, biphasic or other multi-phasic cardiac pacing signal.

To initiate a pacing pulse, control circuit 80 may switch on selected switches in bypass circuit 156 and low side switches 182a, 182b or 182c of HV output circuit 160 upon expiration of a pacing escape interval, e.g., a lower rate interval, a hysteresis interval, an asystole detection interval, a post-shock pacing interval, or an ATP interval, which may be timed out by timing circuit 90 or by timers included in therapy control circuit 94. In some instances, control circuit 80 may initiate delivery of a cardiac pacing pulse signal via bypass circuit 156 and a portion of HV output circuit 160 that includes at least one of the low side switches 182a-c (and excludes switches 180a-c) in response to detecting a pace triggering event, e.g., a sensed R-wave for synchronizing a leading pacing pulse of an ATP sequence or for triggering a back-up safety pacing pulse.

When a pacing pulse width expires, e.g., as determined by therapy control circuit 94 of control circuit 80 or by a timer included in therapy delivery circuit 84, the bypass circuit 156 (and any enabled low side switches 182a-c) may be disabled to terminate the cardiac pacing pulse. Selected switches of bypass circuit 156 and low side switches 182a-c may be re-enabled by a control signal from control circuit 80 for delivering the next cardiac pacing pulse signal, e.g., upon expiration of the next pacing escape interval or upon detection of a next triggering event, from the voltage output signal 164 of voltage regulator 154.

The current flowing from voltage regulator 154 through selected switches included in bypass circuit 156 and through a cardiac pacing output pathway that includes a return path through at least one of HV output circuit low side switches 182a, 182b or 182c maintains the enabled low side switch 182a, 182b and/or 182c in a conducting state because the switches 182a-c require less operating current than the HV output circuit high side switches 180a-c. In this way, a cardiac pacing pulse can be delivered using coil electrodes 24 and 26 with less current than would be required if the cardiac pacing pulse is delivered to electrode terminal 124 or 126 and respective coil electrode 24 or 26 through the high side switches 180a or 180b of the first portion of HV output circuit 160.

In some examples, housing 15 is used as an active can electrode only during CV/DF shock delivery. In this case, cardiac pacing pulses delivered when bypass circuit 156 is enabled are delivered via a pacing electrode vector between coil electrodes 24 and 26. In other examples, housing 15 may be available for use as a return anode with either or both of coil electrodes 24 and 26 selected as the cathode electrode. In still other examples, housing 15 may be available as the pacing cathode electrode with either or both of coil electrodes 24 and 26 selected as the return anode electrode.

By eliminating high side switches 180a-c of HV output circuit 160 from the cardiac pacing output pathway, cardiac pacing pulses can be delivered via coil electrodes 24 and/or 26 more efficiently (less operating current), potentially at higher rates, and without amplitude dipping (which can occur with high current out of the HV holding capacitor 162) compared to when a cardiac pacing pulse is delivered to the coil electrodes 24 and/or 26 via the high side switches 180a and/or 180b of the first portion of HV output circuit 160. The longevity of power source 98 can be conserved by generating a cardiac pacing pulse signal by a cardiac pacing voltage source that includes voltage regulator 154 and delivering the cardiac pacing pulse signal to selected coil electrode terminals 124 and/or 126 using bypass circuit 156 to utilize the relatively low operating current portion of HV output circuit 160 (e.g., low side switches 182a and 182b) compared to delivering cardiac pacing pulses by discharging HV holding capacitor 162 via the high side switches 180a-c and low side switches 182a-c of HV output circuit 160.

FIG. 5 is a diagram of bypass circuit 156 according to some examples. Bypass circuit 156 may include multiple channels for passing a cardiac pacing pulse signal from a cardiac pacing voltage source, e.g., voltage regulator 162 (FIG. 4), to a pacing electrode vector including at least one high surface area, coil electrode 24 or 26 that is also selectable for delivering a CV/DF shock. In the example shown, three bypass circuit channels are shown for coupling the voltage signal 164 from voltage regulator 162 to a cardiac pacing electrode vector selected from coil electrodes 24, 26 and housing 15. Each channel may include one or more switches for coupling the voltage signal 164 to a selected coil electrode 24, coil electrode 26 or housing 15. In the example, shown, each channel includes a pair of switches, e.g., switches 170a and 170b (collectively channel 170) coupled to electrode terminal 124, switches 172a and 172b (collectively channel 172) coupled to electrode 126, and switches 174a and 174b (collectively channel 174) coupled to electrode terminal 115.

The two switches included in a given channel 170, 172 or 174 may each be individually switched on by a control signal received from control circuit 80 for coupling the voltage signal 164 from voltage regulator 154 to a respective coil electrode terminal 124, coil electrode terminal 126 or housing electrode terminal 115. In a disabled or off state, first switches 170a, 172a and 174a of a given channel prevent the amplitude voltage signal 164 from leaking through the second switches 170b, 172b and 174b, which may be triggered on and/or maintained in a conducting state by a lower current than the first switches 170a, 172a, and 174a. In one example, the first switches 170a, 172a and 174a can be a 10 to 30 V rated p-channel MOSFET, e.g., a 20 V rated p-channel MOSFET.

The second switches 170b, 172b and 174b may be solid-state semiconductor switching devices that provide high voltage protection of the circuitry and components of ICD 14. For example, second switches 170b, 172b and 174b may include one or more FETs, diodes, or other devices that require a relatively low or no trigger current to bias into a conducting state. In one example, second switches 170b, 172b and 174b may be implemented as approximately 10 ohm MOSFETs that conduct the pacing current through a parasitic diode from the MOSFET body to drain. When the patient is exposed to a high voltage, e.g., during CV/DF shock delivery by ICD 14 or by an external defibrillator, current may be induced on conductors extending within lead 16. Current flow into bypass circuit 156 and other ICD circuitry is blocked by second switches 170b, 172b and 174b to protect ICD circuitry during exposure to a high voltage. Each channel 170, 172 and 174 may be biased into a conducting state using a relatively low current, e.g., less than 10 microamps or even less than 1 microamp, for enabling cardiac pacing pulses to be delivered to a selected electrode terminal 124, 126 or 115 in a power efficient manner for cardiac pacing using coil electrodes 24 and 26.

In some examples, ICD 14 may be configured to deliver cardiac pacing pulses using coil electrodes 24 and/or 26 in a selected one of an upper range, an intermediate range and a lower range of pacing pulse voltage amplitudes, e.g., based on the cardiac pacing capture threshold. As described above in conjunction with FIG. 4, control circuit 80 may control charging of HV holding capacitor 162 to a cardiac pacing pulse voltage amplitude in the upper range, e.g., greater than 16 V, greater than 20 V, greater than 30 V or greater than 40 V, and control HV output circuit 160 to deliver cardiac pacing pulses having an upper range voltage amplitude using the H-bridge switching circuitry of output circuit 160.

When the pacing capture threshold is less than the upper range and falls into an intermediate range, control circuit 80 may control charging of HV holding capacitor to an intermediate voltage to enable voltage regulator 154 to generate a cardiac pacing pulse signal as voltage output signal 164 to bypass circuit 156 for delivering a cardiac pacing pulse having a voltage amplitude in an intermediate range, e.g., up to a maximum voltage amplitude available from voltage regulator 154, which may be up to 16 V, up to 18 V, up to 20 V, up to 30 V, or up to 40 V as examples. As shown in FIG. 5, a bypass circuit channel 170, 172, or 174 may pass the intermediate voltage amplitude pacing pulse signal to a selected electrode terminal 124, 126 or 115 for use in delivering pacing pulses using at least one of coil electrode 24 and/or 26. The voltage signal 164 passed to bypass circuit 156 from voltage regulator 154 is referred to as an “intermediate voltage” cardiac pacing pulse signal because voltage regulator 154 can be utilized as the cardiac pacing pulse voltage source when the pacing capture threshold is greater than the maximum voltage amplitude available from LV therapy circuit 102 (FIG. 3) but not greater than the voltage amplitude available from voltage regulator 154. When the pacing capture threshold is greater than the voltage amplitude available from the voltage regulator 154, the HV therapy circuit 100 may deliver the pacing pulses in the upper range via HV output circuit 160 using the charged HV capacitor 162 as the cardiac pacing pulse voltage source.

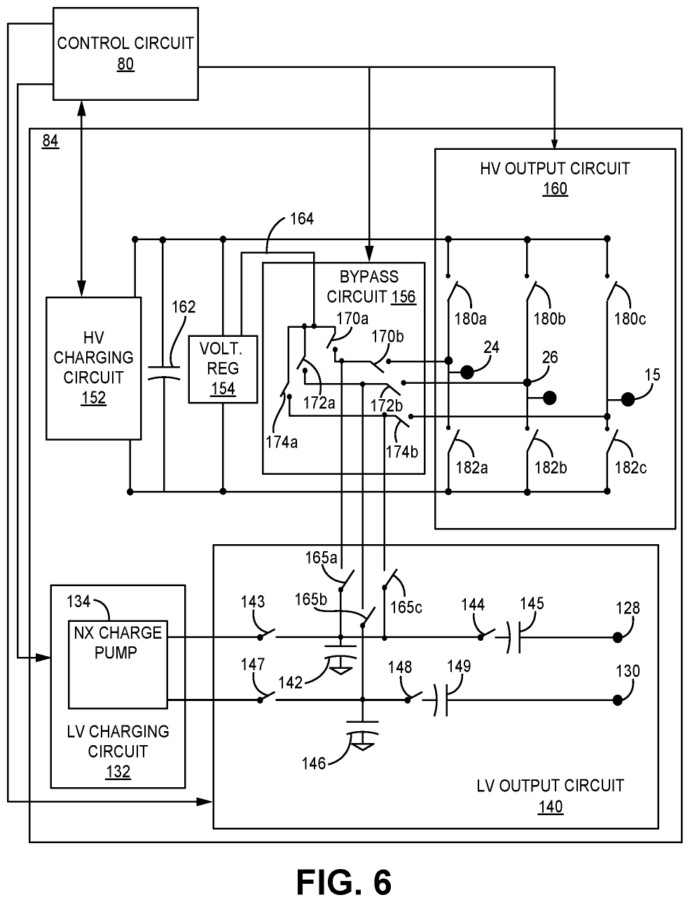

When the pacing capture threshold is in the lower range, bypass circuit 156 may receive a cardiac pacing pulse signal from a cardiac pacing voltage source that is capable of generating pacing pulse signals up to a maximum voltage amplitude of the lower range, e.g., up to 8 V, up to 10 V, up to 12 V or up to 16 V as examples with no limitation intended. Control circuit 80 may enable one of switches 165a, 165b, or 165c for conducting the lower range cardiac pacing pulse signal from a lower range cardiac pacing voltage source to bypass circuit 156. As described below in conjunction with FIG. 6, the LV therapy circuit 102 (shown in FIG. 3) may be configured to generate cardiac pacing pulse signals having a voltage amplitude in the lower range. Bypass circuit 156 may be configured to receive a cardiac pacing pulse signal from the LV therapy circuit 102 at a node between the first switches 170a, 172a, or 174a and the second switches 170b, 172b, or 174b, respectively, of a selected channel of bypass circuit 156 for passing the relatively low amplitude cardiac pacing pulse signal to a selected pacing cathode terminal 124, 126 or 115 via the second switch 170b, 172b, or 174b of a given channel. In this way, bypass circuit 156 may be enabled to deliver cardiac pacing pulse signals in an intermediate range or a lower range of pacing pulse voltage amplitudes using the coil electrodes 24 and/or 26, e.g., in a bipolar pair, with the low side switches 182a-c of HV output circuit 160 for providing a return current path.

FIG. 6 is a conceptual diagram of therapy delivery circuit 84 according to another example. As described above, therapy delivery circuit 84 may include an LV therapy circuit 102, which may include an LV charging circuit 132 and an LV output circuit 140. The LV charging circuit 132 may include one or more charge pumps 134 for charging one of LV holding capacitors 142 or 146 to a pacing voltage amplitude up to a multiple of the battery voltage of power source 98. The charge pump 134 is labeled as an “N×” charge pump because it is capable of charging holding capacitors 142 and 146 up to N times (N×) the battery voltage of power supply 98, where N may be equal to any selected multiple of the battery voltage, e.g., up to two, three or four times the battery voltage. A state machine of control circuit 80 may control charging of a LV holding capacitor 142 or 146 to a programmed pacing voltage amplitude using a multiple of the battery voltage of power source 98. An LV holding capacitor 142 or 146 may have a capacitance of 50 microfarads or less or as low as 10 microfarads or less, as examples.

In some instances, one of ring electrodes 28 or 30 may be selected as the pacing cathode electrode for delivering cardiac pacing pulses. A capacitor selection switch 143 or 147 may be biased to a conducting state by a control signal from control circuit 80 for charging a selected LV holding capacitor 142 or 146 by a charge pump 134 to achieve a desired pacing pulse amplitude in the lower range of pulse voltage amplitudes. The charged holding capacitor 142 or 146 may be discharged via a tip capacitor 145 or 149, respectively, by switching on an electrode selection switch 144 or 148 after charge completion to deliver a pacing pulse to a selected cathode electrode, e.g., ring electrode 28 coupled to electrode terminal 128 or ring electrode 30 coupled to electrode terminal 130. The other ring electrode 30 or 28 may serve as the return anode electrode.

However, when coil electrode 24 and/or coil electrode 26, or in some examples housing 15, is selected as the pacing cathode electrode, control circuit 80 may switch on the corresponding second switch 170b, 172b or 174b to conduct the low amplitude cardiac pacing pulse signal received via one of switches 165a, 165b or 165c from the LV output circuit 140 to the respective electrode terminal 124 and/or 126 (or in some examples housing electrode terminal 115). One of low side switches 182a, 182b or 182c is switched on to provide a return path from a selected pacing anode electrode, e.g., coil electrode 24, coil electrode 25 or housing 15 that is not used as the cathode electrode. Control circuit 80 may select (or a user may program) a cardiac pacing electrode vector that includes coil electrode 24 and/or coil electrode 26. The LV output circuit 140 may pass a cardiac pacing pulse signal via one of switches 165a, 165b or 165c in the lower range of pacing voltage amplitudes to bypass circuit 156 for delivering the lower voltage pacing pulse via at least one or both of coil electrodes 24 and 26 (or housing 15 in some examples). In this way, the second portion of HV output circuit 160, including low side switches 182a-182c, may be used for efficiently delivering lower voltage pacing pulses using coil electrodes 24 and/or 26 without switching on any of high side switches 180a-c.

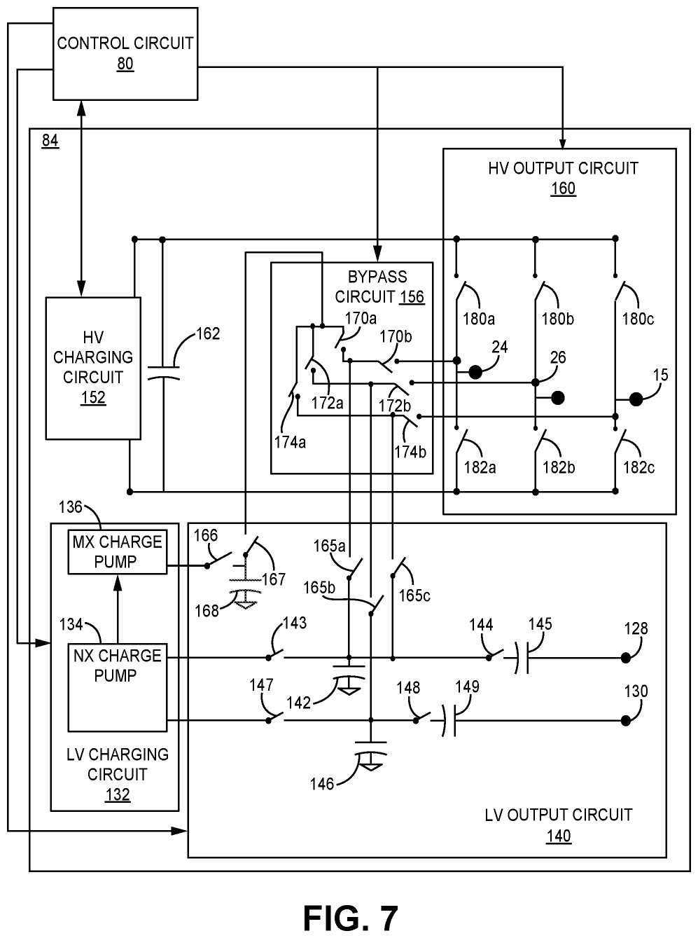

FIG. 7 is a conceptual diagram of therapy delivery circuit 84 according to yet another example. LV therapy circuit 102 may be configured for generating both lower range pacing voltage amplitude signals and intermediate range pacing voltage amplitude signals. For example, LV charging circuit 132 may include multiple charge pumps 134 and 136 for generating cardiac pacing pulse signals in the lower range, e.g., up to 8 V, up to 10 V or up to 12 V as examples using the first charge pump 134. The pacing current can be passed to bypass circuit 156 from LV capacitor 142 or 146 charged by the first charge pump 134. The pacing current can be passed to bypass circuit 156 at the node between a first switch 170a, 172a or 174a and second switch 170b, 172b and 174b, respectively, of a bypass circuit channel for delivering the cardiac pacing pulse via coil electrodes 24 and/or 26 and/or housing 15 and using HV output circuit switches 182a-c for connecting a selected return electrode.

In some examples, LV charging circuit 132 may be selected by control circuit 80 as the cardiac pacing voltage source by enabling multiple charge pumps 134 and 136 to generate cardiac pacing pulse signals received by bypass circuit 156. The cardiac pacing pulses generated using both the first charge pump 134 and the second charge pump 136 in the cardiac pacing voltage source may have an amplitude in the intermediate range of the pacing pulse voltage amplitudes, e.g., between 8 V and 30 V or between 10 V and 30 V or between 10 V and 20 V or between 10 V and 16 V as examples, with no limitation intended. In this case, a holding capacitor 168 may be charged by the output of the second charge pump 136 when switch 166 is enabled by control circuit 80. Holding capacitor 168 may have a higher voltage rating than holding capacitors 142 and 146 but lower than HV holding capacitor 162. Holding capacitor 168 may be charged by the output of the second Mx charge pump 136 when capacitor selection switch 166 is enabled by control circuit 80. The output of the second Mx charge pump 136 may charge holding capacitor 168 up to M times (M×) the output of the first N× charge pump, for a total of M×N times the battery voltage of power source 98. In an illustrative example, the first charge pump 134 is a 3× charge pump and the second charge pump 136 is a 2× charge pump to provide a pacing voltage signal up to 6 times the battery voltage of power source 98.

A terminal of holding capacitor 168, when charged to an intermediate pacing voltage amplitude, can be coupled to the bypass circuit 156 when switch 167 is enabled (and switch 166 is disabled) by control circuit 206. The pacing voltage signal received by bypass circuit 156 can be delivered through a selected channel 170, 172 or 174 of bypass circuit 156 including both the first switch 170a, 172a or 174a and the second switch 170b, 172b or 174b of the selected channel. In this example, control circuit 80 may control therapy delivery circuit 84 to generate and deliver cardiac pacing pulse signals in the intermediate or lower ranges of pacing pulse voltage amplitudes using LV charging circuit 132 and LV output circuit 140 as a cardiac pacing voltage source to deliver ATP during charging of HV holding capacitor 162 for CV/DF shock delivery when a ventricular tachyarrhythmia has been detected by control circuit 80.

In still other examples, therapy delivery circuit 84 may be configured to generate and deliver cardiac pacing pulses having a voltage amplitude in a selected one of multiple intermediate ranges to a pacing electrode vector including coil electrodes 24 and/or 26. For example, using the first N× charge pump 134, which may be a 3× or 4× charge pump as examples, a cardiac pacing pulse signal in the lower range of pacing pulse voltage amplitudes (e.g., up to 8 V or 10 V) may be generated and passed to bypass circuit 156 as shown in FIGS. 6 and 7 and described above. The low amplitude signal may be passed to bypass circuit 156 to a second switch 170b, 172b or 174b of a selected channel 170, 172 or 174 with the first switch 170a, 172a or 174a switched off. Using a combination of the first N× charge pump 134 and the second Mx charge pump 136 a cardiac pacing pulse signal in a first intermediate range of pacing pulse voltage amplitudes (e.g., above the lower range maximum limit of 8 V or 10 V and up to a first intermediate range maximum limit of 16 V to 20 V, as examples) may be generated and passed to bypass circuit 156 to a selected channel 170, 172, or 174 including both the first and second switching channels (both “a” and “b” switches) switched on.

When the pacing capture threshold is greater than the first intermediate range maximum limit, control circuit 80 may control HV charging circuit 152 to generate a rail voltage passed to voltage regulator 154 to generate a cardiac pacing pulse signal having a voltage amplitude in a second intermediate range greater than the first intermediate range. For example, voltage regulator 154 (shown in FIGS. 4 and 6) may pass a cardiac pacing pulse signal 164 to a selected channel 170, 172, or 174 that is in an intermediate range having a minimum voltage limit of 14V, 16 V, 18 V or 20 V, as examples, and a maximum voltage limit of 20 V, 30 V, 40 V or 50 V as examples. If the pacing capture threshold is even higher than a maximum limit of the second intermediate range, control circuit 80 may control HV therapy circuit 100 to generate and deliver cardiac pacing pulses having a voltage amplitude in an upper range using the HV output circuit 160, including selected high side switches 180a-c and selected low side switches 182a-c. The upper range may be greater than the maximum limit of the second intermediate range, e.g., greater than 20 V, greater than 30 V or greater than 40 V and extend up to a maximum allowable pacing pulse volage, e.g., 40 V or 50 V. Thus, control circuit 80 may select from among multiple cardiac pacing voltage sources, which may have over-lapping pacing voltage amplitude ranges, to cover a wide range of available pacing voltage amplitudes from 0.5 V to 50 V, for example, that can be delivered via an appropriate, power efficient output pathway and using coil electrodes 24 and 26.

While not shown in FIG. 7, it is recognized that when therapy delivery circuit 84 includes two or more cardiac pacing pulse voltage sources configured to generate cardiac pacing pulse signals in two or more intermediate ranges of pacing pulse voltage amplitudes, bypass circuit 156 may include additional switches to accommodate each voltage range. For example, each channel 170, 172, and 174 may include a third switch, having a higher voltage rating than the first switches 170a, 172a and 174a, for being switched on by control circuit 80 when voltage regulator passes a cardiac pacing pulse voltage signal 164 in the second intermediate voltage range that is higher than the first intermediate voltage range. In this case, all three switches of a given channel of bypass circuit 156 may be switched on to deliver the cardiac pacing pulse in the second intermediate voltage range to a pacing electrode vector including one or both of coil electrodes 24 and 26. When the pacing voltage amplitude is in the first intermediate range, LV output circuit 140 may pass a cardiac signal pacing pulse signal in the first intermediate range to the node between the third switch (not shown in FIG. 6), which remains switched off, and the first switch 170a, 172a or 174a of the selected channel. The first and second switches of the selected channel 170, 172 or 174 are both switched on to pass the first intermediate range cardiac pacing pulse signal as described above.

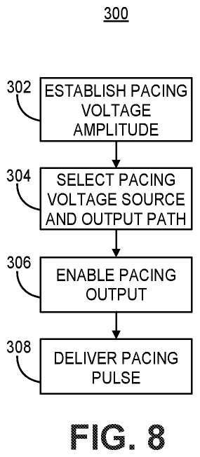

FIG. 8 is a flow chart 300 of a method for delivering cardiac pacing pulses by ICD 14 according to some examples. At block 302, control circuit 80 may establish the pacing voltage amplitude. The pacing voltage amplitude may be established by performing a pacing capture threshold test. The pacing voltage amplitude may be established to be a safety margin (e.g. 0.25 to 5 V or 0.5 to 2 V as examples) greater than a pacing capture threshold determined by the capture threshold test performed by ICD 14. For example, control circuit 80 may initiate a pacing capture threshold test in response to detecting loss of capture or according to a daily or other scheduled capture threshold test protocol. Control circuit 80 may control therapy delivery circuit 84 to deliver a cardiac pacing pulse at one or more pulse voltage amplitudes, which may be tested during delivery to different pacing electrode vectors (e.g., between coil electrodes 24 and 26, between a ring electrode 28 or 30 and a coil electrode 24 or 26, or between ring electrodes 28 and 30) to verify that the heart is captured. Capture may be verified by detecting an evoked response QRS waveform in a cardiac electrical signal sensed by sensing circuit 86 in some examples.

A coil-to-coil pacing electrode vector between coil electrodes 24 and 26 may be used during the pacing capture threshold test and the pacing pulse voltage amplitude may be set based on the determined capture threshold that is the lowest voltage amplitude for a given pulse width that successfully causes myocardial depolarization. In other examples, multiple pacing electrode vectors may be selected from among the available electrodes 24, 26, 28, 30 and 15. The pacing electrode vector associated with the lowest pacing capture threshold may be identified and selected for delivering cardiac pacing pulses with the pacing voltage amplitude established to be a safety margin greater than the pacing capture threshold.

In other examples, the pacing voltage amplitude is established by control circuit 80 based on receipt of a user programmed value via telemetry circuit 88, which may be stored in memory 82. In still other examples, the pacing voltage amplitude may be a default or nominal pacing amplitude that is stored in memory 82. Based on the pacing voltage amplitude established at block 302, control circuit 80 may select a cardiac pacing voltage source and cardiac pacing output pathway at block 304.

Control circuit 80 may compare the established pacing voltage amplitude to a lower range, an intermediate range (or multiple intermediate ranges), and an upper range of pacing voltage amplitudes. The lower, intermediate and upper pacing voltage amplitude ranges may be predefined and stored in memory 82. The lower, intermediate, and upper pacing voltage ranges correspond to the maximum pacing voltage amplitude available from a given cardiac pacing voltage source. For example, with reference to FIG. 6, LV charging circuit 132 may be capable of generating a cardiac pacing pulse signal in the lower range, e.g., up to a maximum of 8 to 10 V, which may include composite pacing pulses as generally disclosed in the above-incorporated U.S. Pat. No. 10,449,362 (Anderson, et al.). Voltage regulator 154 may be capable of passing a voltage signal 164 to bypass circuit 156 as the cardiac pacing pulse signal having an amplitude in an intermediate range, e.g., greater than the maximum limit of the lower range (maximum voltage available from LV therapy circuit 102) and up to 20 V, up to 30 V or up to 40 V in various examples. In other examples, a second charge pump 136 included in LV therapy circuit 102 may be configured to pass a voltage signal to bypass circuit 156 as the cardiac pacing pulse signal having an amplitude in the intermediate range of pacing voltage amplitudes as shown in FIG. 7. HV charging circuit 152 charging HV capacitor 162 may be capable of generating cardiac pacing pulses in an upper range, above the maximum limit of voltage signal 164 output by voltage regulator 154, e.g., greater than 20 V, greater than 30 V or greater than 40 V. Other examples of pacing voltage amplitude ranges are described above, e.g., in conjunction with FIGS. 4-7.

Accordingly, control circuit 80 may select the cardiac pacing voltage source to be received from LV output circuit 140 (utilizing LV charging circuit 132) for a pacing voltage amplitude in the lower range, from voltage regulator 154 (utilizing HV charging circuit 152 and HV holding capacitor 162) or a second charge pump 136 of LV charging circuit 132 and LV output circuit 140 when the pacing voltage amplitude is in an intermediate range, or from HV charging circuit 152 and HV capacitor 162 when the pacing voltage amplitude is in the upper range. Accordingly, the cardiac pacing voltage source of therapy delivery circuit 84 may include multiple, selectable cardiac pacing voltage sources capable of generating pacing pulses in different voltage amplitude ranges.

At block 304, control circuit 80 selects a pacing output pathway based at least in part on the selected voltage source. The pacing output pathway may be selected according to the pacing voltage amplitude and associated pacing pulse voltage source. In some cases, a pacing pathway may be selected in part because it is the most tolerable by the patient and within the limits and capacity of the circuitry of the pacing output pathway. In some cases, control circuit 80 may select the pacing voltage source and output path at block 304 based on the pacing therapy being delivered.

For example, if control circuit 80 detects VT/VF and is controlling therapy delivery circuit 84 to deliver ATP during charging of HV capacitor 162 for shock delivery, control circuit 80 may select the highest intermediate cardiac pacing voltage source available that does not utilize HV capacitor 162. For instance, control circuit 80 may select the output of the second charge pump 136 for charging holding capacitor 168 that can be coupled to a selected channel of bypass circuit 156 for delivering ATP pulses in the intermediate range of the pacing pulse voltage amplitudes. Delivering ATP pulses in the intermediate range promotes a high likelihood of capturing the myocardium via a coil-to-coil pacing electrode vector. ATP delivered during HV capacitor charging via a coil-to-coil pacing electrode vector with ATP pulses having a voltage amplitude in the intermediate range generated by the second charge pump 136 may terminate a VT/VF episode, averting the need for CV/DF shock delivery.

Control circuit 80 may select the output of the voltage regulator 154 or the second charge pump 136 (and holding capacitor 168) coupled to a selected channel of bypass circuit 156 for delivering post-shock pacing pulses or pacing in response to detecting asystole. Pacing pulses having a voltage amplitude to promote a high confidence of successful myocardial capture via a coil-to-coil pacing electrode vector using at least an intermediate pacing voltage amplitude can avoid or prevent asystole. During a post-shock time interval or when asystole is detected by control circuit 80, HV charging circuit 152 may be available for charging HV capacitor 162 to a voltage that is less than the CV/DF shock voltage for enabling voltage regulator 154 to be the cardiac pacing voltage source during post-shock pacing.

At other times, e.g., when bradycardia pacing is being delivered at a lower rate to provide ventricular rate support, control circuit 80 may select the output of the first charge pump 134 for charging a LV holding capacitor 142 or 146 and delivering cardiac pacing pulses in the lower range of voltage amplitudes via the LV output circuit 140 or via bypass circuit 156 and low side switches 182a-c to provide coil-to-coil pacing. Bradycardia pacing at a programmed lower pacing rate may occur over a longer time period than ATP or post-shock pacing and therefore may be more tolerable by the patient when the LV therapy circuit 102 is used for delivering the pacing pulses in a lower pacing pulse voltage amplitude range, which may include composite pacing pulses as described in the above-incorporated U.S. Pat. No. 10,449,362 (Anderson, et al.).

A different cardiac pacing pulse voltage source and pacing output pathway may be selected by control circuit 80, therefore, when the pacing is being delivered for treating a cardiac rhythm that is not imminently life threatening and/or may be sustained for a longer duration of time than the voltage source and output pathway selected by control circuit 80 when the cardiac rhythm is considered more imminently life-threatening and/or delivered over a relatively short duration of time (fewer total cardiac pacing pulses).

When the HV charging circuit 152 and HV capacitor 162 are selected as the pacing voltage source, e.g., due to a pacing voltage amplitude in the upper range, control circuit 80 selects the HV output circuit 160 for the pacing output pathway at block 304. In this case, at block 306, control circuit 80 passes control signals to the HV therapy circuit 100 to control the delivery of one or more pacing pulses at block 308 by charging the HV capacitor 162 to the pacing voltage amplitude and switching on a selected combination of high side switches 180a-c and low side switches 182a-c of HV output circuit 160 at appropriate times for delivering the pacing pulse(s) via coil electrodes 24 and 26 or one of coil electrodes 24 or 26 and housing 15.

When the voltage regulator 154 is selected as the pacing voltage source due to the pacing voltage amplitude being in an intermediate range and/or based on the pacing therapy being delivered, control circuit 80 selects the output pathway to include bypass circuit 156 and the HV output circuit switches 182a-c, excluding the high side switches 180a-c of HV output circuit 160. At block 306, control circuit 80 enables pacing output by passing control signals to HV charging circuit 152 to charge the HV capacitor 162 to a voltage equal to or greater than the pacing voltage amplitude for establishing a positive rail voltage by voltage regulator 154 that may be stepped down to the pacing voltage amplitude as needed. Control circuit 80 passes control signals to bypass circuit 156 and HV output circuit 160 to bias selected switches of bypass circuit 156 and HV output circuit low side switches 182a-c in a conducting state at appropriate times to enable pacing pulse delivery to coil electrodes 24 and 26 or at least one of coil electrodes 24 or 26, e.g., with housing 15 as the return electrode. One or more pacing pulses may be delivered according to the pacing therapy control parameters at block 308 using the voltage regulator 154 as the pacing pulse voltage source and a selected channel of bypass circuit 156, as described above, for excluding the high side switches 180a-c of HV output circuit 160.

When the LV output circuit 134 (utilizing LV charging circuit 132) is selected as the pacing voltage source, control circuit 80 may select bypass circuit 156 and low side switches 182a-c of HV output circuit 160 coupled to coil electrodes 24 and/or 26 as the cardiac pacing output pathway. In some cases, output from the second charge pump 136 in the intermediate range of pacing voltage amplitudes may be passed to bypass circuit and both the first and second switches of a selected channel of bypass circuit 156 may be biased on to conduct the pacing current to a respective pacing electrode terminal. In other instances, the first switches of each channel of the bypass circuit 156 may remain in a non-conducting state. A low voltage cardiac pacing pulse signal may be received from LV therapy circuit 102 by bypass circuit 156 at a node between the first switch and the second switch of a selected channel of bypass circuit 156 for conducting the pacing pulse signal to a selected electrode terminal. The return path is selected by enabling one of the low side HV output circuit switches 182a-c for pacing pulse delivery via one or both coil electrodes 24 and 26. In some instances, LV output circuit 140 coupled to ring electrodes 28 and/or 30 may be selected as the pacing output pathway without using coil electrodes 24 and 26 or any portion of HV output circuit.

The pacing output pathway may be predetermined to be via bypass circuit 156 and a portion of HV output circuit 160 such that at least one coil electrode 24 or 26 is included in the pacing output pathway for all pacing voltage sources. In other examples, the pacing output pathway may be predetermined to be via LV output circuit 140 using ring electrodes 28 and/or 30 when the LV charging circuit 132 provides the cardiac pacing pulse signal for delivering pacing pulses in the lowest range of pacing voltage amplitudes. Bypass circuit 156 and the second portion of HV output circuit 160 (including at least one low side switch 182a, 182b or 182c) may be the predetermined output pathway when the voltage regulator 154 or the second charge pump 136 is selected as the voltage source for providing intermediate pacing pulse voltage amplitudes. In other examples, the cardiac pacing output pathway is selected based on the pacing voltage amplitude and a programmed pacing electrode vector, which may be a coil-to-coil pacing electrode vector between coil electrodes 24 and 26, e.g., as shown in FIG. 1A.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

1-13. (canceled)

14. A medical device comprising a therapy delivery circuit that includes:
a high voltage therapy circuit comprising:
a high voltage capacitor chargeable to a shock voltage amplitude;
a high voltage charging circuit configured to charge the high voltage capacitor to the shock voltage amplitude for generating a cardioversion/defibrillation shock pulse; and
a high voltage output circuit comprising a high side portion configured to couple the high voltage capacitor to a first electrode terminal and a low side portion configured to couple the high voltage capacitor to a second electrode terminal for delivering the cardioversion/defibrillation shock pulse;
a cardiac pacing voltage source configured to generate a cardiac pacing pulse signal having a pacing voltage amplitude that is less than the shock voltage amplitude; and
a bypass circuit configured to couple the cardiac pacing voltage source to a cardiac pacing output pathway that excludes the high side portion of the high voltage output circuit and includes the low side portion of the high voltage output circuit for delivering the cardiac pacing pulse signal via the first electrode terminal and the second electrode terminal.

15. The medical device of claim 14 further comprising:
a sensing circuit configured to sense at least one cardiac signal; and
a control circuit in communication with the sensing circuit and the therapy delivery circuit, the control circuit configured to:
determine a need for cardiac pacing based on the at least one cardiac signal; and
control the therapy delivery circuit to deliver the cardiac pacing pulse signal by enabling the bypass circuit to couple the cardiac pacing voltage source to the cardiac pacing output pathway in response to determining the need for cardiac pacing.

16. The medical device of claim 14 wherein:
the high side portion of the high voltage output circuit comprises:
a first high operating current switching device between a positive terminal of the high voltage capacitor and the first electrode terminal; and
a second high operating current switching device between the positive terminal of the high voltage capacitor and the second electrode terminal; and
the low side portion of the high voltage output circuit comprises:
a third switching device between the first electrode terminal and a negative terminal of the high voltage capacitor; and
a fourth switching device between the second electrode terminal and the negative terminal of the high voltage capacitor, wherein each of the third switching device and the fourth switching device having a low side operating current than the first high operating current switch device and the second high operating current switching device; and
the bypass circuit is configured to couple the cardiac pacing voltage source to the cardiac pacing output pathway that excludes the high side portion of the high voltage output circuit comprising the first switching device and the second switching device.

17. The medical device of claim 14 wherein the bypass circuit comprises at least one bypass switching device; and
the control circuit is further configured to enable the bypass circuit by controlling the at least one bypass switching device to conduct the cardiac pacing pulse signal to the cardiac pacing output pathway.

18. The medical device of claim 14 wherein:
the bypass circuit comprises a first channel including at least a first switching device and a second channel including at least a second switching device; and
the control circuit is configured to selectively enable the first switching device of the first channel or the second switching device of the second channel to conduct the cardiac pacing pulse signal to one of the first electrode terminal or the second electrode terminal, respectively, for bypassing the high side portion of the high voltage output circuit.

19. The medical device of claim 14 further comprising a control circuit configured to enable the bypass circuit for delivering the cardiac pacing pulse; and wherein:
the high voltage charging circuit is configured to generate a rail voltage by charging the high voltage capacitor to a voltage less than the shock voltage amplitude;
the cardiac pacing voltage source further comprises a voltage regulator configured to receive the rail voltage and generate the cardiac pacing pulse signal as a voltage regulated output signal; and
the bypass circuit is configured to, when enabled by the control circuit, couple the voltage regulator to the cardiac pacing output pathway.

20. The medical device of claim 14 further comprising a control circuit configured to enable the bypass circuit for delivering the cardiac pacing pulse; and wherein:
the cardiac pacing voltage source further comprises at least one charge pump for generating the cardiac pacing pulse signal; and
the bypass circuit is configured to, when enabled by the control circuit, couple the cardiac pacing voltage source to the cardiac pacing output pathway by coupling the at least one charge pump to the cardiac pacing output pathway.

21. The medical device of claim 14 wherein:
the cardiac pacing voltage source further comprises:
a first voltage source configured to generate a first cardiac pacing pulse having up to a first maximum voltage amplitude of a first range of pacing pulse voltage amplitudes; and
a second voltage source configured to generate a second cardiac pacing pulse signal having up to a second maximum voltage amplitude of a second range of pacing pulse voltage amplitudes, the second maximum voltage amplitude being greater than the first maximum voltage amplitude; and
the bypass circuit is configured to couple the cardiac pacing voltage source of the therapy delivery circuit to the cardiac pacing output pathway by selectively coupling one of the first voltage source or the second voltage source to the cardiac pacing output pathway.

22. The medical device of claim 21 wherein:
the bypass circuit comprises:
a first channel comprising a first switching device and a second switching device, the second switching device coupled to the first electrode terminal; and
a second channel comprising a third switching device and a fourth switching device, the fourth switching device coupled to the second electrode terminal; and
the medical device further comprising a control circuit that is configured to:
establish a cardiac pacing pulse voltage amplitude;
compare the cardiac pacing pulse voltage amplitude to the first range of pacing pulse voltage amplitudes and the second range of pacing pulse voltage amplitudes;
select one of the first voltage source and the second voltage source based on the cardiac pacing pulse voltage amplitude falling into one of the respective first range of pacing pulse voltage amplitudes and the second range of pacing pulse voltage amplitudes,
in response to selecting the first voltage source, enable one of the second switching device of the first channel or the fourth switching device of the second channel to conduct the first cardiac pacing voltage signal to the respective one of the first terminal or the second terminal; and
in response to selecting the second voltage source, enable one of:
(a) the first switching device and the second switching device of the first channel, or
(b) the third switching device and the fourth switching device of the second channel
to conduct the second cardiac pacing voltage signal to the respective one of the first electrode terminal or the second electrode terminal.

23. The medical device of claim 14 wherein:
the first electrode terminal is couplable to a first high surface area electrode and the second terminal is couplable to a second high surface area electrode for delivering a cardioversion/defibrillation shock pulse; and
the bypass circuit is configured to couple the cardiac pacing voltage source to the cardiac pacing output pathway for delivering the cardiac pacing pulse via the first electrode terminal coupled to the first high surface area electrode and the second electrode terminal coupled to the second high surface area electrode.

24. A method comprising:
generating a cardiac pacing pulse signal by a cardiac pacing voltage source;
enabling a bypass circuit to couple the cardiac pacing voltage source to a cardiac pacing output pathway that excludes a high side portion of a high voltage output circuit of the therapy delivery circuit configured to couple a high voltage capacitor of the therapy delivery circuit to a first electrode terminal for delivering a cardioversion/defibrillation shock pulse and includes a low side portion of the high voltage output circuit configured to couple the high voltage capacitor to a second electrode terminal used for delivering the cardioversion/defibrillation shock pulse; and
delivering the cardiac pacing pulse signal via the first electrode terminal and the second electrode terminal.

25. The method of claim 24, further comprising:
sensing at least one cardiac signal;
determining a need for cardiac pacing based on the at least one sensed cardiac signal; and
in response to determining the need for cardiac pacing, enabling the bypass circuit to couple the cardiac pacing voltage source to the cardiac pacing output pathway.

26. The method of claim 24 further comprising enabling the bypass circuit to couple the cardiac pacing voltage source to the cardiac pacing output pathway by:
excluding the high side portion by excluding, from the cardiac pacing output pathway, a first high operating current switching device between a positive terminal of a high voltage capacitor of the therapy delivery circuit and a first electrode terminal and a second high operating current switching device between the positive terminal of the high voltage capacitor and a second electrode terminal, the high voltage capacitor chargeable to a shock voltage amplitude; and
including the low side portion by including, in the cardiac pacing output pathway, at least one of a third switching device between the first electrode terminal and a negative terminal of the high voltage capacitor and a fourth switching device between the second electrode terminal and the negative terminal of the high voltage capacitor, wherein each of the third switching device and the fourth switching device having a lower operating current than the first high operating current switch device and the second high operating current switching device.

27. The method of claim 24 wherein enabling the bypass circuit comprises controlling at least one switching device of the bypass circuit to conduct the cardiac pacing pulse signal to the cardiac pacing output pathway.

28. The method of claim 24 wherein enabling the bypass circuit further comprises selectively enabling at least one switching device of one of a first channel of the bypass circuit or a second channel of the bypass circuit to conduct the cardiac pacing pulse signal via the at least one switching device to one of the first electrode terminal or the second electrode terminal, respectively, for bypassing the high side portion of the high voltage output circuit.

29. The method of claim 24 further comprising:
generating the cardiac pacing pulse signal by:
generating a rail voltage by charging the high voltage capacitor to a voltage less than the shock voltage amplitude;
receiving the rail voltage by a voltage regulator; and
generating the cardiac pacing pulse signal as a voltage regulated output signal of the voltage regulator; and
enabling the bypass circuit to couple the cardiac pacing voltage source to the cardiac pacing output pathway by enabling the bypass circuit to couple the voltage regulator to the cardiac pacing output pathway.

30. The method of claim 24 further comprising:
generating the cardiac pacing pulse signal by at least one charge pump; and
enabling the bypass circuit to couple the cardiac pacing voltage source to the cardiac pacing output pathway by coupling the at least one charge pump to the cardiac pacing output pathway.

31. The method of claim 14 wherein:
generating the cardiac pacing pulse signal comprises one of:
generating, by a first voltage source of the cardiac pacing voltage source, the cardiac pacing pulse signal as a first cardiac pacing pulse signal having up to a first maximum voltage amplitude of a first range of pacing pulse voltage amplitudes; and
generating, by a second voltage source of the cardiac pacing voltage source, the cardiac pacing pulse signal as a second cardiac pacing pulse signal having up to a second maximum voltage amplitude of a second range of pacing pulse voltage amplitudes, the second maximum voltage amplitude being greater than the first maximum voltage amplitude; and
enabling the bypass circuit to couple the cardiac pacing voltage source to the cardiac pacing output pathway by selectively coupling one of the first voltage source or the second voltage source to the cardiac pacing output pathway.

32. The method of claim 31 wherein:
the bypass circuit comprises:
a first channel comprising a first switching device and a second switching device, the second switching device coupled to a first electrode terminal;
a second channel comprising a third switching device and a fourth switching device, the fourth switching device coupled to a second electrode terminal; and
the method further comprises:
establishing a cardiac pacing pulse voltage amplitude;
comparing the cardiac pacing pulse voltage amplitude to the first range of pacing pulse voltage amplitudes and the second range of pacing pulse voltage amplitudes;
selecting one of the first voltage source and the second voltage source based on the cardiac pacing pulse voltage amplitude falling into one of the respective first range of pacing pulse voltage amplitudes or the second range of pacing pulse voltage amplitudes,
when the first voltage source is selected, enabling one of the second switching device of the first channel or the fourth switching device of the second channel to conduct the first cardiac pacing voltage signal to the respective one of the first electrode terminal or the second electrode terminal; and
when the second voltage source is selected, enabling one of:
(a) the first switching device and the second switching device of the first channel, or
(b) the third switching device and the fourth switching device of the second channel
to conduct the second cardiac pacing voltage signal to the respective one of the first electrode terminal or the second electrode terminal.

33. The method of claim 14 further comprising:
delivering a cardioversion/defibrillation shock pulse via the first electrode terminal coupled to a first high surface area electrode and the second terminal coupled to a second high surface area electrode; and
delivering the cardiac pacing pulse signal when the bypass circuit is enabled via the first electrode terminal coupled to the first high surface area electrode and the second terminal coupled to the second high surface area electrode.

34. A non-transitory, computer readable medium storing a set of instructions that, when executed by a control circuit of a medical device, cause the medical device to:
generate a cardiac pacing pulse signal by a cardiac pacing voltage source of a therapy delivery circuit of the medical device;
enable a bypass circuit of the medical device to couple the cardiac pacing voltage source to a cardiac pacing output pathway that excludes a high side portion of a high voltage output circuit of the therapy delivery circuit configured to couple a high voltage capacitor of the therapy delivery circuit to a first electrode terminal for delivering a cardioversion/defibrillation shock pulse and includes a low side portion of the high voltage output circuit configured to couple the high voltage capacitor to a second electrode terminal used for delivering the cardioversion/defibrillation shock pulse; and
deliver the cardiac pacing pulse signal via the first electrode terminal and the second electrode terminal.
